SEQUENCE LISTING (77 sequences)

SEQ ID NO: 1 - DNA, 16 bp, Artificial sequence
  Synthetic oligonucleotide
ctatataagcagagct

SEQ ID NO: 2 - DNA, 13 bp, Artificial sequence
  Synthetic oligonucleotide
ccctatcagtgat

SEQ ID NO: 3 - DNA, 16 bp, Artificial sequence
  Synthetic oligonucleotide
atcagtgatagagaac

SEQ ID NO: 4 - protein, 34 aa, Artificial sequence
  Synthetic peptide
LTPEQVVAIASNGGGKQALETVQRLLPVLCQAHG

SEQ ID NO: 5 - DNA, 11 bp, Artificial sequence
  Synthetic oligonucleotide
tcttaggtcgt

SEQ ID NO: 6 - DNA, 11 bp, Artificial sequence
  Synthetic oligonucleotide
nnnnnnnnnnn

SEQ ID NO: 7 - DNA, 123 bp, Artificial sequence
  Synthetic oligonucleotide
gccagcaacgatggcggcaagcaagcgctcgaaacggtgcagcggctgttgccggtgctgtgccaggaccatggcctgaccccggaccaagtggtggctatcgccagcaacaatggcggcaag

SEQ ID NO: 8 - DNA, 12 bp, Artificial sequence
  Synthetic oligonucleotide
agcaacgatggc

SEQ ID NO: 9 - DNA, 11 bp, Artificial sequence
  Synthetic oligonucleotide
tatgtgaggag

SEQ ID NO: 10 - DNA, 11 bp, Artificial sequence
  Synthetic oligonucleotide
gttgcctcccc

SEQ ID NO: 11 - DNA, 11 bp, Artificial sequence
  Synthetic oligonucleotide
tgaggagctga

SEQ ID NO: 12 - DNA, 11 bp, Artificial sequence
  Synthetic oligonucleotide
atacaggatga

SEQ ID NO: 13 - DNA, 11 bp, Artificial sequence
  Synthetic oligonucleotide
ttgcctccccc

SEQ ID NO: 14 - DNA, 11 bp, Artificial sequence
  Synthetic oligonucleotide
taataatctga

SEQ ID NO: 15 - DNA, 11 bp, Artificial sequence
  Synthetic oligonucleotide
tttctaggtat

SEQ ID NO: 16 - DNA, 11 bp, Artificial sequence
  Synthetic oligonucleotide
ttcctatgagt

SEQ ID NO: 17 - DNA, 11 bp, Artificial sequence
  Synthetic oligonucleotide
rrrcwwgyyyn

SEQ ID NO: 18 - DNA, 14 bp, Artificial sequence
  Synthetic oligonucleotide
nnnnrrrcwwgyyy

SEQ ID NO: 19 - DNA, 14 bp, Artificial sequence
  Synthetic oligonucleotide
nnnrrrcwwgyyyn

SEQ ID NO: 20 - DNA, 14 bp, Artificial sequence
  Synthetic oligonucleotide
nnrrrcwwgyyynn

SEQ ID NO: 21 - DNA, 14 bp, Artificial sequence
  Synthetic oligonucleotide
nrrrcwwgyyynnn

SEQ ID NO: 22 - DNA, 14 bp, Artificial sequence
  Synthetic oligonucleotide
rrrcwwgyyynnnn

SEQ ID NO: 23 - DNA, 589 bp, Artificial sequence
  Synthetic oligonucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 24 - DNA, 1212 bp, Artificial sequence
  Synthetic oligonucleotide
ggcctccgcgccgggttttggcgcctcccgcgggcgcccccctcctcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatgcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacga

SEQ ID NO: 25 - DNA, 14 bp, Artificial sequence
  Synthetic oligonucleotide
atataaggacgcgc

SEQ ID NO: 26 - DNA, 250 bp, Artificial sequence
  Synthetic oligonucleotide
cgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgt

SEQ ID NO: 27 - DNA, 250 bp, Artificial sequence; identical to SEQ ID NO: 26
  Synthetic oligonucleotide

SEQ ID NO: 28 - DNA, 35 bp, Artificial sequence
  Synthetic oligonucleotide
gtgccacctggtcgacatcgattattgactagatc

SEQ ID NO: 29 - DNA, 35 bp, Artificial sequence
  Synthetic oligonucleotide
gatctagtcaataatcgatgtcgaccaggtggcac

SEQ ID NO: 30 - DNA, 37 bp, Artificial sequence
  Synthetic oligonucleotide
cagtacggtacccggccgcgactctagatcataatca

SEQ ID NO: 31 - DNA, 39 bp, Artificial sequence
  Synthetic oligonucleotide
cagtacgcggccgcgattatgatcagttatctagatccg

SEQ ID NO: 32 - DNA, 37 bp, Artificial sequence
  Synthetic oligonucleotide
cagtacagatcttctcacggcttccctcccgaggtgg

SEQ ID NO: 33 - DNA, 37 bp, Artificial sequence
  Synthetic oligonucleotide
cagtacgtcgacttagacgttgatcctggcgctggcg

SEQ ID NO: 34 - DNA, 37 bp, Artificial sequence
  Synthetic oligonucleotide
cagtacatcgattagttattaatagtaatcaattacg

SEQ ID NO: 35 - DNA, 38 bp, Artificial sequence
  Synthetic oligonucleotide
cagtacatcgatgttaagatacattgatgagtttggac

SEQ ID NO: 36 - DNA, 37 bp, Artificial sequence
  Synthetic oligonucleotide
cagtacggtaccgcgggcccgggatccaccggatcta

SEQ ID NO: 37 - DNA, 39 bp, Artificial sequence
  Synthetic oligonucleotide
cagtacgcggccgcgtcgactgcagaattcctcacgaca

SEQ ID NO: 38 - DNA, 37 bp, Artificial sequence
  Synthetic oligonucleotide
cagtactctagagagctccacttagacggcgaggacg

SEQ ID NO: 39 - DNA, 33 bp, Artificial sequence
  Synthetic oligonucleotide
ccagtatctagacccaccgtactcgtcaattcc

SEQ ID NO: 40 - DNA, 37 bp, Artificial sequence
  Synthetic oligonucleotide
cagtactctagaccaaaaaagaagagaaaggtcgacg

SEQ ID NO: 41 - DNA, 36 bp, Artificial sequence
  Synthetic oligonucleotide
ccagtatctagaaactgatgatttgatttcaaatgc

SEQ ID NO: 42 - DNA, 33 bp, Artificial sequence
  Synthetic oligonucleotide
ccagtatctagattattggccgctggagctgat

SEQ ID NO: 43 - DNA, 33 bp, Artificial sequence
  Synthetic oligonucleotide
ccagtatctagaatggtgtttccttctgggcag

SEQ ID NO: 44 - DNA, 37 bp, Artificial sequence
  Synthetic oligonucleotide
ctagctggtaccctctagatcataatcagcctcgagc

SEQ ID NO: 45 - DNA, 40 bp, Artificial sequence
  Synthetic oligonucleotide
ctagctgcggccgccaagcttatcgatcaaatgtggtatg

SEQ ID NO: 46 - DNA, 31 bp, Artificial sequence
  Synthetic oligonucleotide
ctagctggtacctgatcctctagaccgcttg

SEQ ID NO: 47 - DNA, 34 bp, Artificial sequence
  Synthetic oligonucleotide
ctagctgcggccgccgtggactccaagctggaca

SEQ ID NO: 48 - DNA, 45 bp, Artificial sequence
  Synthetic oligonucleotide
agcttcacaaattcggttctacagggtacacaaattcggttctac

SEQ ID NO: 49 - DNA, 47 bp, Artificial sequence
  Synthetic oligonucleotide
taccctgtagaaccgaatttgtgtaccctgtagaaccgaatttgtga

SEQ ID NO: 50 - DNA, 53 bp, Artificial sequence
  Synthetic oligonucleotide
agggtacacaaattcggttctacagggtacacaaattcggttctacagggtag

SEQ ID NO: 51 - DNA, 51 bp, Artificial sequence
  Synthetic oligonucleotide
gatcctaccctgtagaaccgaatttgtgtaccctgtagaaccgaatttgtg

SEQ ID NO: 52 - DNA, 43 bp, Artificial sequence
  Synthetic oligonucleotide
agcttaacccatggaattcagttctcaaacccatggaattcag

SEQ ID NO: 53 - DNA, 45 bp, Artificial sequence
  Synthetic oligonucleotide
tgagaactgaattccatgggtttgagaactgaattccatgggtta

SEQ ID NO: 54 - DNA, 51 bp, Artificial sequence
  Synthetic oligonucleotide
ttctcaaacccatggaattcagttctcaaacccatggaattcagttctcag

SEQ ID NO: 55 - DNA, 49 bp, Artificial sequence
  Synthetic oligonucleotide
gatcctgagaactgaattccatgggtttgagaactgaattccatgggtt

SEQ ID NO: 56 - DNA, 37 bp, Artificial sequence
  Synthetic oligonucleotide
cagtactccggatctcacggcttccctcccgaggtgg

SEQ ID NO: 57 - DNA, 54 bp, Artificial sequence
  Synthetic oligonucleotide
cagtacctcgaggattatgatctagagtcttagacgttgatcctggcgctggcg

SEQ ID NO: 58 - DNA, 35 bp, Artificial sequence
  Synthetic oligonucleotide
cagtacggtaccgcgccagcgccaggatcaacgtc

SEQ ID NO: 59 - DNA, 42 bp, Artificial sequence
  Synthetic oligonucleotide
cagtacgcggccgcgatcagttatctagatccggtggatcct

SEQ ID NO: 60 - DNA, 31 bp, Artificial sequence; identical to SEQ ID NO: 46
  Synthetic oligonucleotide

SEQ ID NO: 61 - DNA, 34 bp, Artificial sequence; identical to SEQ ID NO: 47
  Synthetic oligonucleotide

SEQ ID NO: 62 - DNA, 37 bp, Artificial sequence
  Synthetic oligonucleotide
cagtactctagaatgaagctactgtcttctatcgaac

SEQ ID NO: 63 - DNA, 37 bp, Artificial sequence
  Synthetic oligonucleotide
cagtactctagactagagattcgtgggggactcgagg

SEQ ID NO: 64 - DNA, 37 bp, Artificial sequence; identical to SEQ ID NO: 56
  Synthetic oligonucleotide

SEQ ID NO: 65 - DNA, 37 bp, Artificial sequence
  Synthetic oligonucleotide
cagtactctagattagacgttgatcctggcgctggcg

SEQ ID NO: 66 - DNA, 37 bp, Artificial sequence
  Synthetic oligonucleotide
cagtactctagaatggcggcgactactgccaaccccg

SEQ ID NO: 67 - DNA, 37 bp, Artificial sequence
  Synthetic oligonucleotide
cagtactctagactatgtaggccgtggttcttggcta

SEQ ID NO: 68 - DNA, 132 bp, Artificial sequence
  Synthetic oligonucleotide
aacgatatgggctgaatacaaaaacgatatgggctgaatacaaaaacgatatgggctgaatacaaaccgcttgaagtctttaattaaaccgcttgaagtctttaattaaaccgcttgaagtctttaattaaa

SEQ ID NO: 69 - DNA, 88 bp, Artificial sequence
  Synthetic oligonucleotide
cgccaatatttacgtgctgctacgccaatatttacgtgctgctacgccaatatttacgtgctgctacgccaatatttacgtgctgcta

SEQ ID NO: 70 - DNA, 92 bp, Artificial sequence
  Synthetic oligonucleotide
ctacctgcactgtaagcactttgctacctgcactgtaagcactttgctacctgcactgtaagcactttgctacctgcactgtaagcactttg

SEQ ID NO: 71 - DNA, 66 bp, Artificial sequence
  Synthetic oligonucleotide
ccgcttgaagtctttaattaaaccgcttgaagtctttaattaaaccgcttgaagtctttaattaaa

SEQ ID NO: 72 - DNA, 92 bp, Artificial sequence
  Synthetic oligonucleotide
cacaaattcggttctacagggtacacaaattcggttctacagggtacacaaattcggttctacagggtacacaaattcggttctacagggta

SEQ ID NO: 73 - DNA, 88 bp, Artificial sequence
  Synthetic oligonucleotide
aacccatggaattcagttctcaaacccatggaattcagttctcaaacccatggaattcagttctcaaacccatggaattcagttctca

SEQ ID NO: 74 - DNA, 20 bp, Artificial sequence
  Synthetic oligonucleotide
aatcccatcaccatcttcca

SEQ ID NO: 75 - DNA, 20 bp, Artificial sequence
  Synthetic oligonucleotide
tggactccacgacgtactca

SEQ ID NO: 76 - DNA, 20 bp, Artificial sequence
  Synthetic oligonucleotide
ctccacttagacggcgagga

SEQ ID NO: 77 - DNA, 20 bp, Artificial sequence
  Synthetic oligonucleotide
gaagtcggccgtatccagag

(19) United States
(12) Patent Application Publication
(10) Pub. No.: US 2016/0369268 A1
(43) Pub. Date: Dec. 22, 2016

(54) TRANSCRIPTION ACTIVATOR-LIKE EFFECTOR (TALE) LIBRARIES AND METHODS OF SYNTHESIS AND USE

(71) Applicant: THE BOARD OF REGENTS OF THE UNIVERSITY OF TEXAS SYSTEMS, Austin, TX (US)

(72) Inventors: Leonidas G. BLERIS, Allen, TX (US); Yi LI, Dallas, TX (US)

(73) Assignee: The Board of Regents of the University of Texas System, Austin, TX (US)

(21) Appl. No.: 14/902,350

(22) PCT Filed: Jun. 25, 2014
(86) PCT No.: PCT/US2014/044032

§ 371 Date: Dec. 31, 2015

Related U.S. Application Data
(60) Provisional application No. 61/841,677, filed on Jul. 1, 2013.

(51) Int. Cl. C12N 15/10

(52) U.S. Cl. CPC C12N 15/1082; C12N 15/1086; C12N 15/1079

(57) ABSTRACT

Disclosed herein are transcription activator-like effector (TALE) libraries that consist of all possible combinations of tandem repeats and methods of making and using the same.

This application claims benefit of priority to U.S. Provisional Application Ser. No. 61/841,677, filed Jul. 1, 2013, the entire contents of which are hereby incorporated by reference.

The invention was made with government support under Grant No. R21GM098984 awarded by the National Institutes of Health and Grant No. CBNET-1105524 awarded by the National Science Foundation. The government has certain rights in the invention.

### BACKGROUND OF THE INVENTION

1. Field of the Invention

The present invention relates generally to the field of biology. More particularly, it concerns transcription activator-like effector (TALE) libraries and methods of making and using the same.

2. Description of Related Art

Transcription activator-like effectors (TALEs) are a new class of specific DNA binding proteins, first discovered in plant pathogenic bacteria Xanthomonas. All naturally occurring TALEs contain a central domain of tandem, 33-35 amino acid repeats, followed by a single truncated repeat of 20 amino acids. Each repeat is largely identical except for two highly variable amino acids at positions 12 and 13, the repeat variable di-residues (RVDs). Recent studies revealed that four most common RVDs each preferentially bind to one of the four bases. This straightforward TALE-DNA binding specificity provides important new tools for genome engineering and targeting. To date, TALEs have been demonstrated to introduce targeted genome modifications (TALE nucleases), induce the expression of specific genes (TALE-VP64) and accomplish the suppression of target genes (TALE-KRAB).

### SUMMARY OF THE INVENTION

In one embodiment, the present invention provides a method of preparing a random N-mer transcription activator-like effector (TALE) library. Said method comprises (a) generating N populations of DNA binding repeats, each comprising repeat variable diresidues (RVDs) flanked by an upstream and a downstream sequence for BsaI-based digestion, wherein the upstream and the downstream flanking sequences for Bsa1-based digestion are unique for each population; (b) digesting the N populations of DNA binding repeats with Bsa1, wherein the resulting 3′ overhang of a first population of DNA binding repeats is complementary to the resulting 5′ overhang of a second population of DNA binding repeats; (c) digesting a plasmid with Bsa1, wherein the resulting 3′ overhang is complementary to the 5′ overhang of the first population of DNA binding repeats and the 5′ overhang is complementary to the 3′ overhang of the N^{th} population of DNA binding repeats; and (d) ligating the digested N populations of DNA binding repeats into the digested plasmid, thereby preparing a random N-mer TALE library. In certain aspects, N may be at least 10. In certain aspects, the plasmids are viral vectors and the library is a viral library.

In certain aspects, the method may further comprise (e) replicating the plasmids within a population of host cells; (f) isolating plasmid DNA from the population of host cells; and (g) pooling the isolated plasmid DNA.

In one aspect, the RVDs in each population of DNA binding repeats may be present in an equal ratio and each module has an equal chance of incorporation. In a further aspect, the random N-mer TALE library may be a balanced library targeting all possible combinations with equal probability. In another aspect, the RVDs in each population of DNA binding repeats may be present in an unequal ratio.

In some aspects, the random N-mer TALE library may be as a nucleotide-biased library. In one aspect, the nucleotide-biased library may be a GC-biased library. In another aspect, the nucleotide-biased library may be an AT-biased library.

In one aspect, select populations of DNA binding repeats may comprise a single RVD. In this and other aspects, the random N-mer TALE library may be a sequence-biased library.

In some aspects, the RVDs may determine the recognition of a base in the target DNA sequence, wherein each DNA binding repeat may be responsible for recognizing one base in the target DNA sequence, and wherein each RVD may comprise a member selected from the group consisting of: NG for recognizing T; HD for recognizing C; NI for recognizing A; NN for recognizing G; and H* for recognizing methylated cytosine (5mC), wherein the * indicates that the second amino acid in the RVD is deleted.

In certain aspects, the random N-mer TALE library may be fused to a nucleotide sequence coding for a functional domain. In some aspects, the functional domain may be a transcription regulatory domain, nuclease, integrase, or nickase. In one aspect, the transcription regulatory domain may be a transcription activator. In another aspect, the transcription regulatory domain may be a transcription repressor.

In one embodiment, the present invention provides a method of determining a TALE that binds to a given nucleotide sequence comprising: (a) obtaining a random N-mer TALE library of the present embodiments; (b) expressing the library in a population of cells that comprise a reporter gene operably linked to a promoter comprising the given nucleotide sequence, wherein expression of the reporter gene is dependent on the presence of a TALE-transcription activator fusion that can bind to the given nucleotide sequence; (c) selecting for cells that express the reporter gene; (d) isolating plasmid DNA from the selected cells; and (e) sequencing the plasmid DNA to determine the sequence of the TALE that bound the given nucleotide sequence.

In one aspect, the given nucleotide sequence may be a promoter. In a further aspect, the promoter may be an endogenous human promoter.

In one embodiment, the present invention provides a method of performing a genetic screen comprising: (a) obtaining a random N-mer TALE library of the present embodiments; (b) expressing the library if step (b) in a population of cells; (c) selecting for cells with a desired phenotype; (d) isolating plasmid DNA from the selected cells; and (e) sequencing the plasmid DNA to determine the sequence of the TALE-fusion that imparted the desired phenotype.

In one aspect, the genetic screen may be performed in yeast. In another aspect, the genetic screen may be a positive genetic screen. In yet another aspect, the genetic screen may be a negative genetic screen.

In one aspect, the screen is performed in human cells. In one aspect, the screen may be a methylation-based genetic screen.

In one aspect, the screen is performed for production of induced pluripotent stem cells.

In one embodiment, the present invention provides a random N-mer TALE library produced according to the methods of the present embodiments.

In one embodiment, the present invention provides a population of host cells comprising a random N-mer TALE library of the present embodiments.

In one embodiment, the present invention provides a method of constructing an N-mer TALE library where each module has an equal chance of incorporation resulting in a balanced library targeting all possible combinations with equal probability.

In one embodiment, the present invention provides a method of constructing an N-mer TALE library where the distribution of the four modules is controlled resulting in a nucleotide-biased library (e.g., GC-rich library).

In one embodiment, the present invention provides a method of constructing a constrained N-mer TALE library where specific positions are fixed and others are selected according to the input distribution resulting in a target sequence-biased library that will, for example, target a specific motif.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C: Design and conceptual elements of the TALE-hybrid approach. (FIG. 1A) Schematic of general TALE design guidelines in this study. TALE hybrids were designed to control expression of target genes, and their functions regulated by small molecules or endogenous signals. (FIG. 1B) Illustration of TALE-based rewiring of endogenous signals to chromosomal gene expression. (FIG. 1C) Schematic illustration of the stably integrated AmCyan transcript. The DNA binding sequences for three TALEs included in this study are shown.

FIGS. 2A-C: TAL effectors controlling the expression of the AmCyan transgene cassette. (FIG. 2A) Induction of expression of AmCyan fluorescent proteins by TALE_{TRE}-VP16 activators. Different amounts of these two TALE fusion constructs were transiently transfected. 48 hours post transfection, cells were subjected to flow cytometry analysis. The fluorescence readings of wells which received control vector were used as the baseline and were subtracted from all other experimental samples. Bar graphs show expression levels of AmCyan as determined by flow cytometry and represent average and standard deviation from three replicates. TALE_{TRE#3} and TALE_{TRE#4} were designed to target the TRE sequence and fused with VP16 transactivation domain. Left, top: Both TALE_{TRE}-VP16 fusion proteins strongly induced the expression of AmCyan. Right, top: Fluorescence microscopy images of TALE-induced AmCyan expression in selected samples (I-IV). Bottom: 3-D illustration of overlaid flow cytometry histograms of AmCyan expression under the induction of TALE_{TRE}-VP16. I, control vector (250 ng); II, TALE_{TRE#3}-VP16 (250 ng); III, TALE_{TRE#4}-VP16 (250 ng); IV, TALE_{TRE#3}-VP16 (125 ng)+TALE_{TRE#4}-VP16 (125 ng). (FIG. 2B) Induction of expression of AmCyan fluorescent proteins by TALE_{TRE}-p65 activators. Left: Bar graphs representing expression levels of AmCyan as determined by flow cytometry. Left, inserts: Fluorescence microscopy images of selected samples (II, III). Right: Overlaid flow cytometry histograms of AmCyan expression between samples (II, III) and sample I (control). I, control vector (250 ng); II, TALE_{TRE#3}-p65 (250 ng); III, TALE_{TRE#4}-p65 (250 ng). (FIG. 2C) Suppression of expression of AmCyan fluorescent proteins by TALE_{TRE}-KRAB transcriptional repressors. The cells were induced by 10 ng TALE_{TRE#4}-VP16 and were co-transfected with different amounts of TALE_{TRE#3}-KRAB, TALE_{TRE#4}-KRAB, or TALE_{CMV}-KRAB plasmids. 72 hours post transfection the cells were subjected to the same analysis as above. The fluorescence readings of wells which received no TALE_{TRE#4}-VP16 induction were used as the baseline and were subtracted from all other experimental samples. Top: Fluorescence microscopy images of AmCyan expression in selected samples (I-VI). Middle: Bar graphs representing expression levels of AmCyan as determined by flow cytometry. Bottom: Overlaid flow cytometry histograms of AmCyan expression between samples (II-VI, filled histograms) and sample I (positive control, black line histogram).

FIG. 3: Schematic illustration of TRE DsRed monomer and U6 shRNA-FF3 transcripts.

FIGS. 4A-B: TALE_{CMV}-KRAB suppressed the expression of mKate fluorescent reporter gene under the control of CMV promoter. Different amounts of TALE_{CMV}-KRAB were co-transfected with CMV-mKate-PEST. (FIG. 4A) Bar graphs showing expression of mKate. (FIG. 4B) Overlaid flow cytometry histograms of mKate expression between 0 ng of TALE_{CMV}-KRAB and 10 ng of TALE_{CMV}-KRAB (left), or 300 ng of TALE_{CMV}-KRAB (middle), or negative control (0 ng of CMV-mKate-PEST, right).

FIGS. 5A-B: Suppression of expression of AmCyan fluorescent proteins by TALE_{TRE}-KRAB transcriptional repressors. TALE_{TRE#3}-KRAB, TALE_{TRE#4}-KRAB, and TALE_{CMV}-KRAB were transiently transfected into TRE_AmCyan HEK293 stable cells. 24 hours post transfection cells were induced with 0.3 mg/ml doxycycline. The cells were then subjected to flow cytometry analysis after an additional 48 hours. The fluorescence readings of wells which received no doxycycline induction were used as the baseline and were subtracted from all other experimental samples. All experiments were performed in triplicates. (FIG. 5A) Bar graphs representing expression levels of AmCyan as determined by flow cytometry. (FIG. 5B) Fluorescence microscopy images of AmCyan expression in selected samples (I-IV). I, 0.3 μg/ml doxycycline+empty vector (400 ng); II, 0.3 μg/ml doxycycline+TALE_{CMV}-KRAB (400 ng); III, 0.3 μg/ml doxycycline+TALE_{TRE#3}-KRAB (400 ng); IV, 0.3 μg/ml doxycycline+TALE_{TRE#4}-KRAB (400 ng).

FIG. 6: Competitive inhibitory effects between TALE_{TRE}-KRAB and TALE_{TRE}-VP16. The TRE_AmCyan HEK293 cells were co-transfected with 150 ng of TALE_{TRE}-KRAB and different amounts of TALE_{TRE#4}-VP16. Cells which received only 200 ng of TALE_{TRE#4}-VP16 were used as the positive control. 72 hours post transfection, cells were subjected to fluorescence microscopy and flow cytometry analysis. The fluorescence readings of wells which received control vector were used as the negative control and were subtracted from all other experimental samples. All experiments were performed in triplicates. (Top). Bar graphs representing expression levels of AmCyan as determined by flow cytometry. TALE_{TRE#4}-VP16 partially counteracts the inhibitory effects of TALE_{TRE}-KRAB in a dose-dependent manner. (Top, inlets). Fluorescence microscopy images of AmCyan expression in selected samples (I-VII). (Bottom). 3-D illustration of overlaid flow cytometry histograms of AmCyan in cell samples transfected with different amounts of TALE_{TRE#4}-VP16. (front to back: 0, 75 and 200 ng of TALE_{TRE#4}-VP16, positive control). I, TALE_{TRE#3}-KRAB (150 ng)+TALE_{TRE#4}-VP16 (0 ng); II, TALE_{TRE#3}-KRAB (150 ng)+TALE_{TRE#4}-VP16 (75 ng); III, TALE_{TRE#3}-KRAB (150 ng)+TALE_{TRE#4}-VP16 (200 ng); IV, TALE_{TRE#4}-VP16 (200 ng); V, TALE_{TRE#4}-KRAB (150 ng)+TALE_{TRE#4}-VP16 (0 ng); VI, TALE_{TRE#4}-KRAB (150 ng)+TALE_{TRE#4}-VP16 (75 ng); VII, TALE_{TRE#4}-KRAB (150 ng)+TALE_{TRE#4}-VP16 (200 ng).

FIGS. 7A-C: Induction of expression of AmCyan fluorescent proteins by TALE-based two hybrid system. (FIG. 7A) Schematic illustration of the TALE-based two-hybrid method. The TALE_{TRE#3} and TALE_{TRE#4} were fused with Rheo Receptor. 500 ng of these two TALE fusion plasmids were co-transfected into the cells with 500 ng of EF1-Rheo Activator. The cells were treated with different concentration of GenoStat ligand. 72 hours post transfection the cells were subjected to fluorescence microscopy and flow cytometry analysis. All experiments were performed in triplicates. (FIG. 7B) 3-D illustration of overlaid flow cytometry histograms of AmCyan in cell samples treated with different concentration of GenoStat. (front to back: 0, 4, 20, 100 and 500 nM of GenoStat). (FIG. 7C) Fluorescence microscopy images and bar graph representations of AmCyan expression in same samples. The AmCyan signals correlate with GenoStat concentrations.

FIGS. 8A-E: TALE interface with endogenous transcription factor and microRNA signals. (FIG. 8A) The 1-454 amino acids of human ARNT protein fused to the TALE_{TRE#3} and TALE_{TRE#4} DNA binding domains reacting with HIF-1α under hypoxic conditions and inducing the transgene amCyan. (FIG. 8B) Induction of expression of AmCyan fluorescent proteins by TALE_{TRE}-ARNT1-454 fusions under treatment of CoCl₂ (100 μM). The TALE_{TRE#3} and TALE_{TRE#4} were fused with amino acids 1-454 of human ARNT protein. 800 ng of these two TALE fusion plasmids were transfected into the cells with or without treatment of CoCl₂ (100 μM). 72 hours post transfection the cells were subjected to flow cytometry analysis. All experiments were performed in triplicates. Overlaid flow cytometry histograms of AmCyan in cell samples with or without CoCl₂ treatment. Incubation with 100 μM of CoCl₂ significantly increased the expression of AmCyan fluorescent protein. Bar graphs represent AmCyan expression in same samples. TALE_{TRE#4}-Rheo Receptor was included as the negative control. (FIG. 8C) MiR-16 and miR-17 target sequences incorporated into 3′-UTR regions of TALE_{TRE#3}-VP16 and TALE_{TRE#4}-VP16 constructs. FF4 targets are used as the negative control. 10 ng of each construct were transiently transfected into cells. 72 hours post transfection cells were subjected to fluorescence microscopy and flow cytometry analysis. The fluorescence readings of wells which received control vector were used as the baseline and were subtracted from all other experimental samples. All experiments were performed in triplicates. (FIG. 8D) Overlaid flow cytometry histograms of AmCyan between samples with (filled histograms) or without (black line histograms) miR-16 or -17 target sequences. (FIG. 8E) Fluorescence microscopy images and bar graphs showing relative mRNA level of TALE-VP16 and expression level of AmCyan signals. The induction capacity of TALE_{TRE}-VP16 was significantly lower when miR-16 or -17 targets were inserted. ** denotes p<0.01.

FIG. 9: Suppression of TALE_{TRE}-VP16-dependent expression of AmCyan by miRFF4. MiR-FF4 target sequences were incorporated into 3′-UTR regions of TALE_{TRE#3}-VP16 and TALE_{TRE#4}-VP16 constructs. 10 ng of each of such constructs were transiently transfected into TRE_AmCyan HEK293 stable cells with or without 100 ng of EF1-Neo-FF4. 72 hours post transfection, cells were subjected to fluorescence microscopy and flow cytometry analysis. The fluorescence readings of wells which received control vector were used as the baseline and were subtracted from all other experimental samples. All experiments were performed in triplicates. (Top) Fluorescence microscopy images and bar graphs showing expression level of AmCyan signals. The induction capacity of TALE_{TRE}-VP16 was significantly lower when EF1-Neo-FF4 were co-transfected. ** denotes p<0.01. (Bottom) Overlaid flow cytometry histograms of AmCyan between samples with or without co-transfection of EF1-Neo-FF4.

FIGS. 10A-B: Suppression of TALE_{TRE}-VP16-dependent expression of AmCyan by endogenous miRNAs. MiR-17, miR-10b and miR-146a target sequences were incorporated into 3′-UTR regions of TALE_{TRE#3}-VP16 and TALE_{TRE#4}-VP16 constructs. 10 ng of each of such constructs were transiently transfected into TRE_AmCyan HEK293 stable cells. 72 hours post transfection, cells were subjected to fluorescence microscopy and flow cytometry analysis. The fluorescence readings of wells which received control vector were used as the baseline and were subtracted from all other experimental samples. All experiments were performed in triplicates. (FIG. 10A). Bar graphs showing expression levels of AmCyan signals. * and ** denote p<0.05 and p<0.01, respectively. (FIG. 10B). Overlaid flow cytometry histograms of AmCyan between samples (II-IV) and sample I, as well as samples (VI-VIII) and sample V. I, TALE_{TRE#3}-VP16; II, TALE_{TRE#3}-VP16-4XmiR-17tgts; III, TALE_{TRE#3}-VP16-4XmiR-10btgts; IV, TALE_{TRE#3}-VP16-4XmiR-146atgts; V, TALE_{TRE#4}-VP16; VI, TALE_{TRE#4}-VP16-4XmiR-17tgts; VII, TALE_{TRE#4}-VP16-4XmiR-10btgts; VIII, TALE_{TRE#4}-VP16-4XmiR-146atgts.

FIGS. 11A-C: Construction of an 11-mer TALE-VP16 library. (FIG. 11A) Schematic illustration of a TALE protein depicting the tandem repeat domain and the variable diresidues (RVDs). (FIG. 11B) Schematic illustration of a typical TALE assembling reaction. Corresponding RVDs were chosen for specific nucleotide targets (NI for A, HD for C, NG for T, and NN for G). (FIG. 11C) Schematic illustration of the TALE library assembling. For each position, equal amounts of all four building modules were used, which results in an 11-mer TALE library covering all possible 11-mer DNA targets.

FIG. 12A-C: Test of library quality by Sanger sequencing. (FIG. 12A) The sequencing profile of the inventors' 11-mer TALE library. There are 6-nucleotide long repeats (RVDs), spaced by 102 nucleotides, showing “noisy” signals; (FIG. 12B) Expected nucleotide compositions of the RVD domain of the inventors' TALE library; (FIG. 12C) Observed nucleotide compositions of the RVD domain of the inventors' TALE library.

FIGS. 13A-F: Isolation of TALE-VP16 fusions targeting the human SCN9A gene using the 11-mer TALE-VP16 library and the yeast one-hybrid assay. (FIG. 13A) Schematic illustration of the yeast one-hybrid assay using the 11-mer TALE-VP16 library. A bait sequence was cloned in front of an antibiotic resistance gene (Aba resistance gene) in yeast. The 11-mer TALE-VP16 library was then transformed into this stable clone and a surviving assay was performed on -Leu plates containing 100 nM Aba. (FIG. 13B) The RVD sequences of isolated TALE-VP16 fusions and their targets within the human SCN9A bait sequence (scale not proportional). TALE-VP16 fusions were shown to bind to both the plus and the minus strands of the SCN9A bait sequence. (FIG. 13C) The isolated TALE-VP16 fusions induced overexpression of endogenous SCN9A in HEK293 cells and A431 cells. The mRNA levels of SCN9A were determined by quantitative RT-PCR. An empty vector (PEF-1) was used as the control. Columns 1-6: All TALE-VP16 fusions were able to effectively induce the overexpression of SCN9A in HEK293 cells (n=5). Columns 11-16: All TALE-VP16 fusions effectively induced the overexpression of SCN9A in A431 cells (n=3). Columns 7-10: All 4 TALEs designed according to TALE-NT 2.0 failed to induce the overexpression of SCN9A in HEK293 cells (n=3). Inlet: Western blot shows that all TALE-VP16 fusions induced the overexpression of SCN9A protein in A431 cells (representative data of two independent experiments). (FIG. 13D) The RVD sequences of isolated TALE-VP16 fusions and their targets within the human miR-34b/c bait sequence (scale not proportional). (FIG. 13E) Confirmation of the binding between isolated TALE-VP16 fusion M1 and its predicted gene target within the human miR-34b/c bait sequence. The isolated clone M1 was predicted to target 5′-TTTCTAGGTAT-3′ within the miR-34b/c bait sequence. The full-length bait sequence (pAbAi-miR-34b/c) or bait with the predicted target site deleted (pAbAi-miR-34b/c (ATTTCTAGGTAT)) was stably integrated into yeast cells. TALE-VP16 fusion clone M1 was then transformed into either cell line. Only cells which contained the intact bait sequence survived the 100 nM Aba selection. (FIG. 13F) The isolated TALE-VP16 fusion M1 effectively induced overexpression of miR-34b in a dose-dependent manner in both HEK293 and HeLa cells (n=3 for both cell lines).

FIGS. 14A-C: Genetic screen for cycloheximide resistance in yeast using the TALE-VP16 plasmid library. (FIG. 14A) Confirmation of the genuine positive yeast clones conferring cycloheximide resistance. 18 positive clones were isolated from the cycloheximide resistance screening. Subsequently, these positive TALE-VP16 fusion plasmids were recovered and again re-transformed into the wild-type yeast cells. The transformed cells were then re-streaked onto -Leu plates containing 0.5 ng/ml of cycloheximide. After 3 days, cells transformed with genuine positive clones pGADT7-TALE-A8-VP16 and pGADT7-TALE-A35-VP16 were able to grow robustly. In contrast, cells transformed with the false positive clone pGADT7-TALE-A12-VP16 or the control pGADT7 failed to grow. (FIG. 14B) The isolated TALE-VP16 fusion clones A8 and A35 bind to the promoters of the PDR3 and PDR5 genes. TALE-VP16 fusion clones A8 and A35 were isolated from cycloheximide resistance screening. Both A8 and A35 were predicted to bind to the promoter of PDR3 gene. In addition, A35 was predicted to target the promoter of the PDR5 gene. Four copies of the predicted PDR3/PDR5 promoter targets and their immediate adjacent sequences were cloned in front of a fluorescence reporter gene (mKATE2) in yeast (bait). These yeast stable clones were then transformed with corresponding pGADT7-TALE-A8-VP16, pGADT7-TALE-A35-VP16 or pGADT7 (control). TALE-VP16 fusion clones A35 or A8 potently induced the expression of mKATE2 in yeast cells containing the corresponding baits, while pGADT7 failed to do so (right). (FIG. 14C) The isolated TALE-VP16 fusions A8 and A35 induced overexpression of endogenous PDR3 and PDR5 genes. Wild-type yeast cells were transformed with pGADT7-TALE-A8-VP16, pGADT7-TALE-A35-VP16 or pGADT7 (control). The expression levels of PDR3/PDR5 were measured by quantitative RT-PCR. Both clones were able to effectively induce the overexpression of PDR3 (n=3) and PDR5 (n=3).

FIGS. 15A-E: Sanger sequencing profiles of the p53-biased 11-mer TALE library. (FIG. 15A) The nucleotide compositions of the variable diresidues (RVDs) for four target nucleotides (T, C, A, G). (FIG. 15B) The expected nucleotide compositions of the RVD domains for various target nucleotides (R, W, Y, N). (FIG. 15C) Observed nucleotide compositions of the first four RVD domains of the p53-biased 11-mer TALE library using the forward primer (P23), which closely tracks the predicted composition. (FIG. 15D) Observed nucleotide compositions of the last two RVD domains of the p53-biased 11-mer TALE library using the reverse primer (P24), which closely tracks the predicted composition. (FIG. 15E) Since TALE binding sites are preferentially preceded by a T, five 14-mer TALE-VP16 libraries which target 5′-NNNNRRRCWWGYYY-3′, 5′-NNNRRRCWWGYYYN-3′,5′-NNRRRCWWGYYYNN-3′,5′-NRRRCWWGYYYNNN-3′, and 5′-RRRCWWGYY-3′ can be prepared separately. Pooling these five libraries is predicted to cover at least 1-(0.75)⁵=75% of all possible 14-mer DNA target sequences which contain a p53-responsive element and are preceded by a T.

FIGS. 16A-B: Genetic screening of amCyan overexpression in human HEK293 cells using the TALE-VP16 AAV viral library. A HEK293 stable cell line harboring a TRE-amCyan stable integration was infected with the 11-mer TALE-VP16 AAV viral library at various MOIs (400, 120, 40 and 0). 48 hours later, the cells were subjected to fluorescence microscopy and flow cytometry. Cells receiving only complete growth medium were used as the negative control, while cells treated with 1 ug/mL of DOX were used as the positive control. (FIG. 16A) Fluorescence microscopy images of TALE-induced amCyan expression in a subpopulation of cells infected with the 11-mer TALE-VP16 viral library at various MOIs. (FIG. 16B) Overlaid flow cytometry histograms of amCyan expression in the negative control, the positive control and cells infected with the 11-mer TALE-VP16 viral library at various MOIs.

### DESCRIPTION OF ILLUSTRATIVE EMBODIMENTS

Transcription activator-like effectors (TALEs) are a new class of specific DNA binding proteins, first discovered in plant pathogenic bacteria Xanthomonas. The inventors conceived and implemented a new way to build TALE libraries. Specifically, the inventors modified the Golden Gate assembly method to construct a 11-mer TALE library which covers all possible 11-mer DNA targets (4¹¹=4,194,304). The consistency of this library was confirmed by Sanger sequencing.

The inventors applied a TALE-VP16 library (V 16 is an activation functional domain) to a yeast one-hybrid assay to select for the TALEs with strongest binding. Specifically, the inventors cloned part of the 5′-UTR and the ORF of human SCN9A gene in front of an antibiotic resistance gene in yeast. The inventors were able to identify and isolate five TALE-VP16 clones, which were then verified that drive the overexpression of endogenous SCN9A in human cells, based on quantitative RT-PCR assay (up to 11-fold increase).

The implications of this general technology can be immense for developing new generations of genetic screens. The inventors argue that the TALE libraries (coupled with a functional domain) will be superior to current technologies and hold significant commercial potential. Key advantages of the technology include: 1. The ability to apply both negative and positive action in genetic screens using the corresponding functional domain. 2. The modularity of the functional domain allows for applications other than transcriptional control. For example, theoretically it can be fused with methylases or integrases domains. 3. The ability to introduce multiple rounds of positive and negative screening, and importantly the combination between coupled positive and/or negative action. 4. The size of the DNA target can be controlled at the cost of increasing the size of the library. Increasing the DNA target size will result to superior specificity, in other words will reduce the cross-talk of the TALEs (seemingly the main drawback of RNAi-based genetic screens). 5. The ability to target genomic areas of specific nucleotide content (e.g. GC rich) by producing a TALE library enriched in these nucleotides (biased libraries). 6. The TALE protein coding sequences can be isolated and sequenced, which will facilitate the rapid identification of their target genes. This is especially advantageous compared to other genomic screening approaches (e.g. mutagenesis, CRISPR/Cas system).

### I. TAL EFFECTORS

Recent advancements in genome editing tools enable targeted, sequence-specific modification and regulation of gene networks. Transcription activator-like effectors, proteins secreted by Xanthomonas plant pathogenic bacteria, have been a major breakthrough in the rapid and systematic synthesis of these editing tools to target any DNA sequence of choice (Cermak et al., 2011; Bogdanove and Voytas, 2011; Boch and Bonas, 2010; Zhang et al., 2011). Efforts in developing TAL effector technology have led to applications, such as activation, repression, deletion, and insertion of a target gene, that are currently expanding to a wide range of model organisms and cell types (Reyon et al., 2012; Marx, 2012; Li et al., 2011; Li et al., 2011b; Mahfouz et al., 2011; Mahfouz and Li, 2011; Maresca et al., 2012; Mercer et al., 2012; Sun et al., 2012; Tesson et al., 2011). At the industrial scale, binary logic analogs could assist in managing population heterogeneities and processing environmental signals such as the oxygen level in a bioreactor (Shiue and Prather, 2012). Additionally, this progress may lead to rapid means for prototyping modified pathways and exploring control of endogenous transcripts within chromosomes (Li et al., 2012).

TAL effectors have a modular DNA-binding domain (DBD); each repeat region consists of 34 amino acids (Kay et al., 2007; Munoz Bodnar et al., 2012). A pair of residues at the 12^{th} and 13^{th} position of each repeat region determines the nucleotide specificity and is referred to as the repeat variable diresidue (RVD) (Boch et al., 2009; Mak et al., 2012). The last repeat region, termed the half-repeat, is typically truncated to 20 amino acids (Bogdanove and Voytas, 2011). Combining these repeat regions creates the potential to synthesize sequence-specific synthetic TALEs (Li et al., 2012; Garg et al., 2012; Carlson et al., 2012). The C-terminus has a nuclear localization signal (NLS) which directs a TALE towards the nucleus once it enters a cell, and an acidic activation domain (AD) which increases gene expression (Boch and Bonas, 2010; Schornack et al., 2008; Gurlebeck et al., 2005; Kay et al., 2005). The endogenous NLS is often replaced by an organism-specific localization signal. For example, an NLS derived from the simian virus 40 large T-antigen can be used for applications in mammalian cells (Zhang et al., 2011). In application, this activation domain can be replaced by another functional domain to expand the toolbox and allow for more fine-tuned control of genetic networks.

On average, the most efficient TALEs range from 15.5-19.5 repeats (Boch and Bonas, 2010). The repeats HD, NG, NI, and NN are used to target C, T, A, and G/A, respectively (Bogdanove and Voytas, 2011; Cong et al., 2012). Recent studies suggest that NH may have higher specificity for G and promote higher TALE activity (Cermak et al., 2011; Sanjana et al., 2012). This basic code enables DNA targeting where each RVD corresponds to a specific nucleotide (Streubel et al., 2012). Out of the RVDs that have close to a one-to-one correspondence, HD and NN seem to bind more strongly to DNA (though NN has specificity to G/A). To build the most efficient TALEs, it may help to include ˜3-4 stronger RVDs in the TALE array while avoiding more than 6 weaker RVDs in a row, especially at either end of the repeat region (Streubel et al., 2012). However, there are additional TAL repeats that can be used for degenerate TALE-DNA interactions. NS can target A/G/T/G and NK targets G, but seems to have less DNA-binding affinity than NH. Additionally, N*, where * is an RVD with a deletion in the 13^{th} residue, does not seem to have binding specificity or affinity (Streubel et al., 2012), which may help target a methylated cytosine. Further work has also shown that NV, S* and NA have an ability to bind to any DNA nucleotide (Cong et al., 2012).

TALE activity can be modulated by varying the number and composition of repeats within the DNA binding domain(s). Thus, TALEs can be engineered to recognize a DNA sequence of interest by (1) varying the number of repeats to modulate activity, (2) selecting different binding sites to achieve different levels of activity, and (3) varying the composition of RVDs and their fit to the target site.

Methods are provided herein for identifying TAL effectors having enhanced targeting capacity for a target DNA. Such methods can include generating a nucleic acid library encoding TAL effectors that comprises DNA binding domains having a plurality of DNA binding repeats, each repeat containing RVDs that determine recognition of a base pair in the target DNA. The specificities of exemplary RVDs include: NN (G), HD (C), NG (T), NI (A), NS (A or C or G), N* (SmetC), HG (T), H* (T), IG (T), HA (C), ND (C), NK (G), HI (C), HN (G), NA (G), SN (G or A), and YG (T), where the asterisk indicates a gap at the second position of the RVD.

A. Crystal Structure

Two separate groups have helped elucidate the crystal structure of TAL effectors to further understand some aspects of TALE-DNA affinity. Mak, et al. and Deng, et al found HD and NN form stronger interactions with DNA by forming hydrogen bonds. By contrast, weaker domains, such as NG and NI form van der Waals interactions with DNA (Mak et al., 2012; Deng et al., 2012). Deng et al. (2012) examined the crystal structure of dHax3, an artificially synthesized TAL with 11.5 repeats comprising of HD, NG, NS, in the DNA-bound and DNA-free states. The dHax TAL effector has a right-handed superhelical pitch of 60 Å, which is reduced to 35 Å in the DNA bound state; overall, there is a compression of the superhelical structure in the DNA-bound state that adds to the flexibility of TALEs binding to DNA with minor shifts. Mak et al. (2012) investigated the crystal structure of PthXo1, a TAL protein with 23.5 repeats, in its DNA bound state and suggest that a proline at the 27^{th} position of each repeat may be important for the consecutive packing of TAL repeats and for the TAL effector-DNA association. Compared to Deng et al. (2012), Mak et al. (2012) studied a naturally occurring TAL effector, and they were able to analyze a wider variety of RVDs, including HD, NG, NI, NN, NS, “N*”, and NG. Though Deng et al. were limited in the range of repeats they used, their analysis on DNA-bound and DNA-free TALEs is notable.

Both studies from Mak et al. and Deng et al. analyzed TALE crystal structure data of the TALE DNA-binding domain (DBD). Recent work has resolved the crystal structure of the N and C terminus of the TALE protein (Gao et al., 2012). Most importantly, their work shows that residues 162-288 of the N terminus have 4 repeat regions that directly bind to DNA and are structurally similar to the TALE repeats without specificity.

B. Assembly of TALE Proteins

Several kits and commercial solutions allow rapid, custom assembly of TALE repeat regions between the N and C terminus of the protein, which function as a DNA Binding Domain (DBD) (Cermak et al., 2011; Reyon et al., 2012; Marx et al., 2012; Li et al., 2012). These assembly methods synthesize custom DNA binding domains, which are then cloned into an expression vector containing a functional domain. Many of these options for de novo synthesis of TALEs or TALENs in the laboratory combine digestion and ligation steps in a Golden Gate reaction with type II restriction enzymes (Cermak et al., 2011; Sanjana et al., 2012). High-throughput assembly methods of TALE proteins include Ligation-Independent Cloning (LIC), Fast Ligation-based Automatable Solid-phase High-throughput (FLASH) assembly, and Iterative-Capped Assembly (ICA) (Schmid-Burgk et al., 2012). FLASH uses a library of 376 plasmids containing 1-, 2-, 3-, or 4-mers to synthesize up to 96 TALEs in less than a day (Mercer et al., 2012). Alternatively, the iterative capped assembly (ICA) method constructs TALs by sequentially adding monomers to create custom length TAL effectors in parallel without relying on an extensive library (Briggs et al., 2012). A recently developed method, LIC uses larger overhangs (10-30 bp) than Golden-gate based assemblies; these overhangs remain stable during transformation and eliminate the need for a prior ligation step. Furthermore, LIC has high fidelity, eliminating the need for a selection procedure under optimal conditions (Schmid-Burgk et al., 2012).

C. Repression and Activation

The ability to coordinate gene network expression with both activation and repression could expand simultaneous control of multiple genes (Keasling, 2008). In order to reach this goal, stable integrations of TALE activator and repressor proteins under ligand control (Li et al., 2012) may be a useful tool to regulate endogenous genes in a controlled manner. Furthermore, inducible activation under the control of endogenous signaling such as hypoxia or exogenous ligands enables advanced circuit design (Li et al., 2012) and combinations of TAL effectors can help perturb feedback systems within endogenous pathways.

Most repression techniques rely on fusing the TALE with an existing functional domain known to interfere with the RNA Polymerase II complex (Peng et al., 2000). TAL effectors with either the KRAB domain or the mSin3 Interacting Domain decrease mammalian transcription (Cong et al., 2012; Li et al., 2012). Furthermore, TALE repressors in combination with post-transcriptional repressors such as shRNA show near complete repression in mammalian cells (Garg et al., 2012).

Advances in TALE activation (Li et al., 2012) and software for developing orthogonal TALE targets (Garg et al., 2012) will improve the study of important pathways (Keasling, 2012). A combination of TALE activators targeting an endogenous promoter showed strong synergistic effects and increased transcription up to a hundred fold over basal conditions (Maeder et al., 2013). Furthermore, several C termini modifications have shown strong increases in gene expression. Studies have found that only maintenance of around 68 of the C terminus amino acids remain necessary for high fold change in Hax3 TALE activation (Zhang et al., 2011). TALEs with the herpes simplex virus derived VP-64 activation domain (AD) show higher activation with a truncated C terminus than synthetic TALEs retaining the full C terminus with VP-64 added. Weaker activation domains such as the AD of human NF-κB add to the variety of options for gene activation. Taken together, TAL effectors are effective tools for targeted up-regulation or down-regulation of gene expression.

D. Nucleases

TALENs utilize a C-terminal fusion with the type II restriction enzyme Fold to create a heterodimer which produces a double-stranded break (DSB) in DNA (Streubel et al., 2012). Nuclease induced DSBs are repaired by non-homologous end joining (NHEJ) or homologous directed repair (HDR), where homologous recombination (HR) is the most important type of HDR. NHEJ is an error-prone mechanism that results in a functional gene knockout by creating small insertions or deletions (indels) while HR, in combination with a template donor DNA sequence, results in a gene insertion or direct nucleotide exchange (Streubel et al., 2012; Moore, 2012, PloS One).

In some cases, TALENs are more efficient than engineered zinc-fingers in cutting DNA in vivo when injected as mRNA (Tesson et al., 2011). Notably, the majority of newly designed TALENs often show cutting capability (Schmid-Burgk et al., 2012) with one group reporting 87% success rate in de novo TALENs (Reyon et al., 2012), and a rapid ligation independent TALEN construction with success rates as high as 59% in newly targeted sequences and 86% in sequences established as amenable to TALEN cutting.

TALENs have been useful in creating knockout strains and studying mutations in a variety of organisms such as bacteria (Politz et al., 2013), yeast (Cermak et al., 2011; Bogdanove and Voytas, 2011; Li et al., 2011; Li et al., 2011b), plants (Mahfouz et al., 2011; Morbitzer et al., 2010), human cell lines (Zhang et al., 2011; Miller et al., 2010; Geissler et al., 2011; Ding et al., 2012), rodents (Tesson et al., 2011; Wefers et al., 2013), and rat embryonic stem cells (Tong et al., 2012). In addition, TALE nucleases have successfully modified human stem cells, allowing editing and gene expression tools for tissue engineering (Hockemeyer et al., 2011).

Several assays allow researchers to assess the cutting efficiencies of TALENs, which will help in developing new and useful applications (Sanjana et al., 2012; Certo et al., 2011). The surveyor method can be used to detect DSBs by PCR amplification. Another method is the traffic light reporter (TLR) assay, which can be used to determine whether a TALEN cuts the target DNA and induces NHEJ or HR. A mutated GFP and a frameshifted RFP provide the initial target DNA for the TALEN. If HR occurs, a functioning GFP protein replaces the mutated GFP; if NHEJ occurs, red fluorescence protein (RFP) is shifted into frame.

E. Nickases

Though TALE nucleases have tremendous potential, they are more likely to repair a DSB using NHEJ. Both NHEJ and HR are believed to be competing pathways (Hartlerode and Scully, 2009). Error-prone NHEJ is effectively eliminated by TALE nickases. TALE-MutH has recently been shown to be an efficient, programmable nickase (Gabsalilow et al., 2013). Here, single TALE-MutH protein is able to create the desired single-stranded break (SSB) in DNA, thereby inducing the HR repair mechanism. Other strategies to create TALE nickases may involve the Fok1 nuclease, where one unit of the heterodimer is catalytically inactive (Ramirez et al., 2012).

F. Recombinases

Site-specific recombinases (SSRs) can integrate, excise, or invert specified DNA segments. Most SSRs are part of one of two major families: tyrosine (2) recombinases and serine (resolvase/invertase) recombinases. Tyrosine recombinases use a Holliday junction to break and rejoin single strands in pairs while serine recombinases introduce a DSB before strand exchange (Grindley et al., 2006). Mercer et al. (2012) created recombinatorial TALE proteins (TALER) by fusing a Gin invertase, a serine recombinase, to edit both mammalian and bacterial cells at specific locations. This study also shows that longer targets of 26 and 32 by recombined 100 fold more efficiently than the shorter targets of 14 and 20 base pairs in E. coli. Translating this assay to mammalian cells showed a ˜20 fold efficiency with a 44 by target and ˜6 fold efficiency with a 32 by target. However, heterodimers of zinc finger recombinases (ZFRs) and TALERs seemed to rescue recombinase activity. Further studies with TALERs in mammalian cells must be done to explore the full potential of this new technology.

G. Perspectives

TALEs may be promising in addressing current challenges in rewiring and programming endogenous networks to achieve metabolic engineering goals (Pennisi, 2012; Tyo et al., 2007; Yonekura-Sakakibara et al., 2012). This technology provides a framework for modular DNA targeting and standardized assembly methods to rapidly and efficiently test binding sequences of interest. An enormous amount of effort involved the synthesis of a library of 18,740 TALEN pairs to span the human genome. 140 of the TALEN pairs from this library were tested in HEK293 cells, using the T7 endonuclease as an assay to confirm cutting activity (Kim et al., 2013).

Methylation:

Methylation is an important epigenetic process that has a role in major biological processes such as development and cancer gene expression by regulating promoter activity through chromatin modification; in eukaryotes methylation occurs at the cytocine residue (Suzuki and Bird, 2008). Normally, this cytosine is next to a guanine so that 2 diagonal cytosines are methylated. Earlier crystal structure data suggests that both HG and N* do not have an amino acid side chain (Valton et al., 2012), which allows for flexibility in accepting a pyramidine (though this is not highly selective and a purine can be accepted as well (Mak et al., 2012; Bochtler, 2012). Further structural analysis from Deng et al. (2012) suggests that NG may be able to recognize 5-methylcytocine. In the TALE cipher, NG normally targets T, but their experimental data shows that a methylated region is targeted by NG, but not HD. NG has less affinity for mC in the target DNA than an unmethylated region, but NG is significantly stronger at binding mC than C (Deng et al., 2012). However, recent experimental work from Kim, et al. found that TALEs were not as efficient at recognizing methylated DNA target regions (Kim et al., 2013). These data suggest that more work must be done to explore the potential of TALEs targeting methylated CpG sites.

Biofuels:

Synthetic metabolic networks may help in addressing the world's challenges in making efficient and sustainable biofuels (Lee et al., 2008; Clomburg and Gonzalez, 2010). To address this challenge, TALENs should offer a means of prototyping new strains of algae geared toward fuel production. Given the high priority objective within metabolic engineering to maximize biofuel production efficiency (Boyle and Silver, 2011), standardized and high-throughput methods in metabolic and genetic engineering will be critical in optimizing microorganisms to reach the upper limits of biofuel production efficiency (Christi, 2007; Reyon et al., 2011).

Cancer:

Recent advances in the ease of genome sequencing help to lower the cost of personalized medicine and will improve synthetic biology techniques in targeted cancer treatment (Ruder et al., 2011). To enable these goals, TALEs in combination with metabolic engineering techniques may have useful future applications in cancer screening, therapy, and drug production. Many cancers are caused by defects in the DNA damage response. TALE nucleases could also be used as a sensor to detect cancer by creating targeted DSBs; unfitting repair of the DSB would predict a greater likelihood of chromosome instability or cancerous cells (Khanna and Jackson, 2001). As a preventative step, TALENs can be used for targeted gene editing of mutations that have a high likelihood of causing cancer. Additionally, TALE recombinases may be valuable in enabling algae gene manipulation to produce the desired products, considering recent efforts to use algae as a chassis to produce eukaryotic cancer drugs (Tran et al., 2013).

Thus far, TAL effectors have shown tremendous potential in targeted genome editing and regulation. Genome editing tools, in general, are rapidly advancing towards more precision and efficiency. Zinc fingers, TALENs, and CRISPR/Cas9, the newest addition to the toolbox, show potential in rewiring gene networks for both therapeutics and industry.

### II. POLYNUCLEOTIDES AND RECOMBINANT NUCLEIC ACID CONSTRUCTS

The terms “nucleic acid” and “polynucleotide” are used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). PCR refers to a procedure or technique in which target nucleic acids are amplified. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be obtained by mutagenesis. For example, a donor nucleic acid sequence can be mutated using standard techniques, including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992.

Recombinant nucleic acid constructs (e.g., vectors) also are provided herein. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The terms “regulatory region,” “control element,” and “expression control sequence” refer to nucleotide sequences that influence transcription or translation initiation and rate, and stability and/or mobility of the transcript or polypeptide product. Regulatory regions include, without limitation, promoter sequences, enhancer sequences, response elements, protein recognition sites, inducible elements, promoter control elements, protein binding sequences, 5′ and 3′ untranslated regions (UTRs), transcriptional start sites, termination sequences, polyadenylation sequences, introns, and other regulatory regions that can reside within coding sequences, such as secretory signals, Nuclear Localization Sequences (NLS) and protease cleavage sites.

As used herein, “operably linked” means incorporated into a genetic construct so that expression control sequences effectively control expression of a coding sequence of interest. A coding sequence is “operably linked” and “under the control” of expression control sequences in a cell when RNA polymerase is able to transcribe the coding sequence into RNA, which if an mRNA, then can be translated into the protein encoded by the coding sequence. Thus, a regulatory region can modulate, e.g., regulate, facilitate or drive, transcription in a cell, animal, or tissue in which it is desired to express a modified target nucleic acid.

A promoter is an expression control sequence composed of a region of a DNA molecule, typically within 100 nucleotides upstream of the point at which transcription starts (generally near the initiation site for RNA polymerase II). Promoters are involved in recognition and binding of RNA polymerase and other proteins to initiate and modulate transcription. To bring a coding sequence under the control of a promoter, it typically is necessary to position the translation initiation site of the translational reading frame of the polypeptide between one and about fifty nucleotides downstream of the promoter. A promoter can, however, be positioned as much as about 5,000 nucleotides upstream of the translation start site, or about 2,000 nucleotides upstream of the transcription start site. A promoter typically comprises at least a core (basal) promoter. A promoter also may include at least one control element such as an upstream element. Such elements include upstream activation regions (UARs) and, optionally, other DNA sequences that affect transcription of a polynucleotide such as a synthetic upstream element.

The choice of promoters to be included depends upon several factors, including, but not limited to, efficiency, selectability, inducibility, desired expression level, and cell or tissue specificity. For example, tissue-, organ- and cell-specific promoters that confer transcription only or predominantly in a particular tissue, organ, and cell type, respectively, can be used. Other classes of promoters include, but are not limited to, inducible promoters, such as promoters that confer transcription in response to external stimuli such as chemical agents, developmental stimuli, or environmental stimuli.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

A 5′ untranslated region (UTR) is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA message stability or translation attenuation. Examples of 3′ UTRs include, but are not limited to polyadenylation signals and transcription termination sequences. A polyadenylation region at the 3′-end of a coding region can also be operably linked to a coding sequence.

The vectors provided herein also can include, for example, origins of replication, and/or scaffold attachment regions (SARs). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

By “delivery vector” or “delivery vectors” is intended any delivery vector which can be used in the presently described methods to put into cell contact or deliver inside cells or subcellular compartments agents/chemicals and molecules (proteins or nucleic acids). It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.

The terms “vector” or “vectors” refer to a nucleic acid molecule capable of transporting another nucleic acid to which it has been linked. A “vector” in the present document includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Of particular interest for use as a delivery vector, the inventors will utilize adeno-associated virus (AAV), a small virus which infects humans and some other primate species. AAV is not currently known to cause disease and consequently the virus causes a very mild immune response. AAV Vectors can infect both dividing and quiescent cells and persist in an extrachromosomal state without integrating into the genome of the host cell. These features make AAV a very attractive candidate for creating viral vectors for gene delivery. Human clinical trials using AAV for gene therapy in the retina have shown promise. Commercial AAV systems are available from Clontech, Agilent and Vector Systems.

One type of vector is an episome, i.e., a nucleic acid capable of extrachromosomal replication. Preferred vectors are those capable of autonomous replication and/or expression of nucleic acids to which they are linked. Vectors capable of directing the expression of genes to which they are operatively linked are referred to herein as “expression vectors. A vector according to the present document comprises, but is not limited to, a YAC (yeast artificial chromosome), a BAC (bacterial artificial), a baculovirus vector, a phage, a phagemid, a cosmid, a viral vector, a plasmid, a RNA vector or a linear or circular DNA or RNA molecule which may consist of chromosomal, non chromosomal, semi-synthetic or synthetic DNA. In general, expression vectors of utility in recombinant DNA techniques are often in the form of “plasmids” which refer generally to circular double stranded DNA loops which, in their vector form are not bound to the chromosome. Large numbers of suitable vectors are known to those of skill in the art. Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracyclin, rifampicin or ampicillin resistance in E. coli. Preferably said vectors are expression vectors, wherein a sequence encoding a polypeptide of interest is placed under control of appropriate transcriptional and translational control elements to permit production or synthesis of said polypeptide. Therefore, said polynucleotide is comprised in an expression cassette. More particularly, the vector comprises a replication origin, a promoter operatively linked to said encoding polynucleotide, a ribosome binding site, a RNA-splicing site (when genomic DNA is used), a polyadenylation site and a transcription termination site. It also can comprise an enhancer or silencer elements. Selection of the promoter will depend upon the cell in which the polypeptide is expressed. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-3-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

Inducible promoters may be induced by pathogens or stress, more preferably by stress like cold, heat, UV light, or high ionic concentrations (reviewed in Potenza et al. (2004) In vitro Cell Dev Biol 40:1-22). Inducible promoter may be induced by chemicals [reviewed in Moore et al. (2006); Padidam (2003); Wang et al. (2003); and Zuo and Chua (2000)].

Delivery vectors and vectors can be associated or combined with any cellular permeabilization techniques such as sonoporation or electroporation or derivatives of these techniques.

It will be understood that more than one regulatory region may be present in a recombinant polynucleotide, e.g., introns, enhancers, upstream activation regions, and inducible elements.

Recombinant nucleic acid constructs can include a polynucleotide sequence inserted into a vector suitable for transformation of cells (e.g., animal cells). Recombinant vectors can be made using, for example, standard recombinant DNA techniques (see, e.g., Sambrook et al. (1989) Molecular Cloning: A Laboratory Manual, 2nd ed., Cold Spring Harbor Laboratory, Cold Spring Harbor, N.Y.).

A recombinant nucleic acid sequence as described herein can integrate into the genome of a cell via illegitimate (i.e., random, non-homologous, non site-specific) recombination, or a recombinant nucleic acid sequence as described herein can be adapted to integrate into the genome of a cell via homologous recombination. Nucleic acid sequences adapted for integration via homologous recombination are flanked on both sides with sequences that are similar or identical to endogenous target nucleotide sequences, which facilitates integration of the recombinant nucleic acid at the particular site(s) in the genome containing the endogenous target nucleotide sequences. Nucleic acid sequences adapted for integration via homologous recombination also can include a recognition site for a sequence-specific nuclease. Alternatively, the recognition site for a sequence-specific nuclease can be located in the genome of the cell to be transformed. Donor nucleic acid sequences as described below typically are adapted for integration via homologous recombination.

In some embodiments, a nucleic acid encoding a selectable marker also can be adapted to integrate via homologous recombination, and thus can be flanked on both sides with sequences that are similar or identical to endogenous sequences within the plant genome (e.g., endogenous sequences at the site of cleavage for a sequence-specific nuclease). In some cases, nucleic acid containing coding sequence for a selectable marker also can include a recognition site for a sequence-specific nuclease. In these embodiments, the recognition site for the sequence-specific nuclease can be the same as or different from that contained within the donor nucleic acid sequence (i.e., can be recognized by the same nuclease as the donor nucleic acid sequence, or recognized by a different nuclease than the donor nucleic acid sequence).

In some cases, a recombinant nucleic acid sequence can be adapted to integrate into the genome of a cell via site-specific recombination. As used herein, “site-specific” recombination refers to recombination that occurs when a nucleic acid sequence is targeted to a particular site(s) within a genome not by homology between sequences in the recombinant nucleic acid and sequences in the genome, but rather by the action of recombinase enzymes that recognize specific nucleic acid sequences and catalyze the reciprocal exchange of DNA strands between these sites. Site-specific recombination thus refers to the enzyme-mediated cleavage and ligation of two defined nucleotide sequences. Any suitable site-specific recombination system can be used, including, for example, the Cre-lox system or the FLP-FRT system. In such embodiments, a nucleic acid encoding a recombinase enzyme may be introduced into a cell in addition to a donor nucleotide sequence and a nuclease-encoding sequence, and in some cases, a selectable marker sequence. See, e.g., U.S. Pat. No. 4,959,317.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Transcription Activator-Like Effector Hybrids for Conditional Control and Rewiring of Chromosomal Transgene Expression

The ability to conditionally rewire pathways in human cells holds great therapeutic potential. Transcription activator-like effectors (TALEs) are a class of naturally occurring specific DNA binding proteins that can be used to introduce targeted genome modifications or control gene expression. Here, the inventors present TALE hybrids engineered to respond to endogenous signals and capable of controlling transgenes by applying a predetermined and tunable action at the single-cell level. Specifically, the inventors first demonstrate that combinations of TALEs can be used to modulate the expression of stably integrated genes in kidney cells. The inventors then introduce a general purpose two-hybrid approach that can be customized to regulate the function of any TALE either using effector molecules or a heterodimerization reaction. Finally, the inventors demonstrate the successful interface of TALEs to specific endogenous signals, namely hypoxia signaling and microRNAs, essentially closing the loop between cellular information and chromosomal transgene expression.

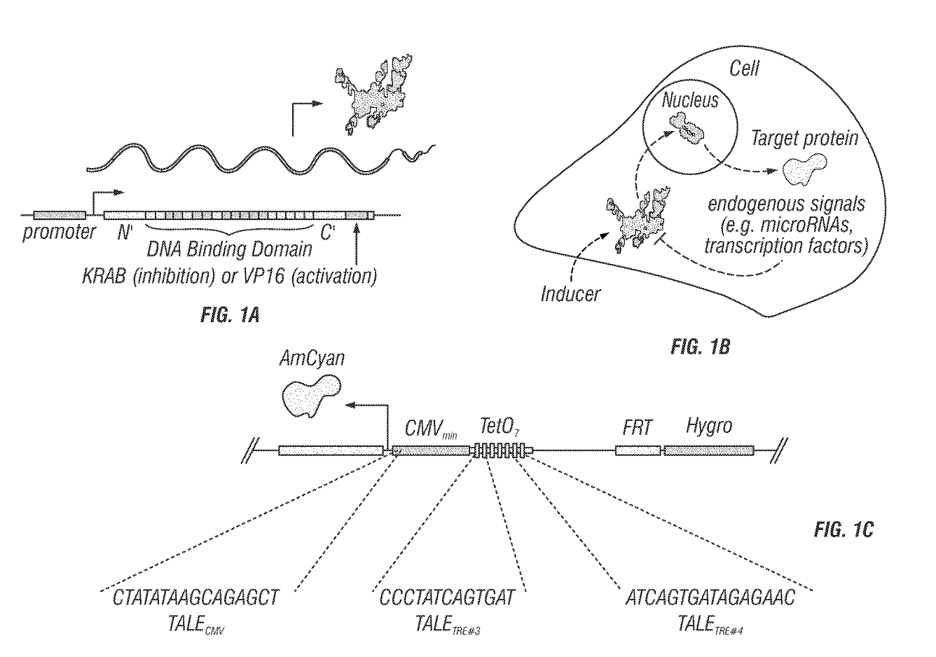

Transcription activator-like effectors (TALEs) were first discovered in plant pathogenic bacteria Xanthomonas (Sugio et al., 2007; Boch et al., 2010). Most naturally occurring TALEs contain a central domain of tandem, 33-35 amino acid repeats, followed by a single truncated repeat of 20 amino acids (FIG. 1a). Each repeat is largely identical except for two variable amino acids at positions 12 and 13, the repeat variable di-residues (RVDs). Protein crystallography (PX) studies reveal that each TALE repeat contains two helices connected by a short RVD-containing loop. The protein forms a right-handed, superhelical structure with RVDs contacting the major groove of the DNA double helix. The 12^{th} residue helps stabilize the RVD loop, while the 13^{th} residue participates in the base-specific contact (Mak et al., 2012; Deng et al., 2012; Bradley et al., 2012). Further studies have shown that the four most common RVDs each preferentially bind to one of the four bases (HD to C, NI to A, NG to T, NN to G) (Moscou and Bogdanove, 2009; Boch et al., 2009; Streubel et al., 2012).

The straightforward TALE-DNA binding specificity provides important new tools for genome engineering and targeting (Briggs et al., 2012; Doyle et al., 2012). TALEs were fused with the catalytic domain of the Fold endonuclease to generate a new class of sequence-specific nucleases, the TAL effector nucleases (TALENs) (Li et al., 2011; Kim et al., 2011; Christian et al., 2010; Kleinstiver et al., 2012). TALENs, when used in pairs, can produce double-strand breaks between the target sequences and induce non-homologous end-joining and homologous recombination in endogenous target genes, such as a mutant form of the human β-globin (HBB) gene associated with sickle cell disease (Sun et al., 2012). Secondly, TALE fusion proteins which contain transactivation domains were generated to induce the expression of specific genes and thus could potentially be used as therapeutic tools for hereditary diseases (Cermak et al., 2011). For example, TALEs which specifically target the human frataxin promoter were fused with VP64 transcription activator, and the resulting fusion increased endogenous frataxin gene expression (Tremblay et al., 2012). Finally, TALEs were fused with the KRAB transcriptional repression domain, and these fusion TALE repressors were able to efficiently repress in transient transfections the synthetic fluorescent reporter gene which contains target sequences of TALEs (Garg et al., 2012), as well as the transcription of endogenous human SOX2 gene (Cong et al., 2012).

Venturing towards rewiring endogenous signals to chromosomal gene expression (FIG. 1b), the inventors first performed a comprehensive characterization of TALE hybrids engineered for transgene activation and repression. Subsequently, based on a 2-hybrid approach, the inventors engineered two different mechanisms to modulate any TALE function in cells. The first system is based on fusing a custom TALE protein to synthetic heterodimers that bind depending on the concentration of an externally delivered effector molecule, and accordingly result to the recruitment of transcriptional components and the initiation of transcription of a target transgene. The second system is based on a fusion of a custom TALE protein to a sequence that forms a heterodimer with an endogenous transcription factor that translocates into the nucleus only under specific cellular conditions, and again results to the initiation of the target transgene transcription. Finally, the inventors successfully interfaced functional TALEs with endogenous microRNAs (miR-16 and miR-17) and a transcription factor (HIF-1α), essentially closing the loop between specific cellular signals and chromosomal gene expression.

Results

Characterization of TALE-Based Activation and Inhibition.

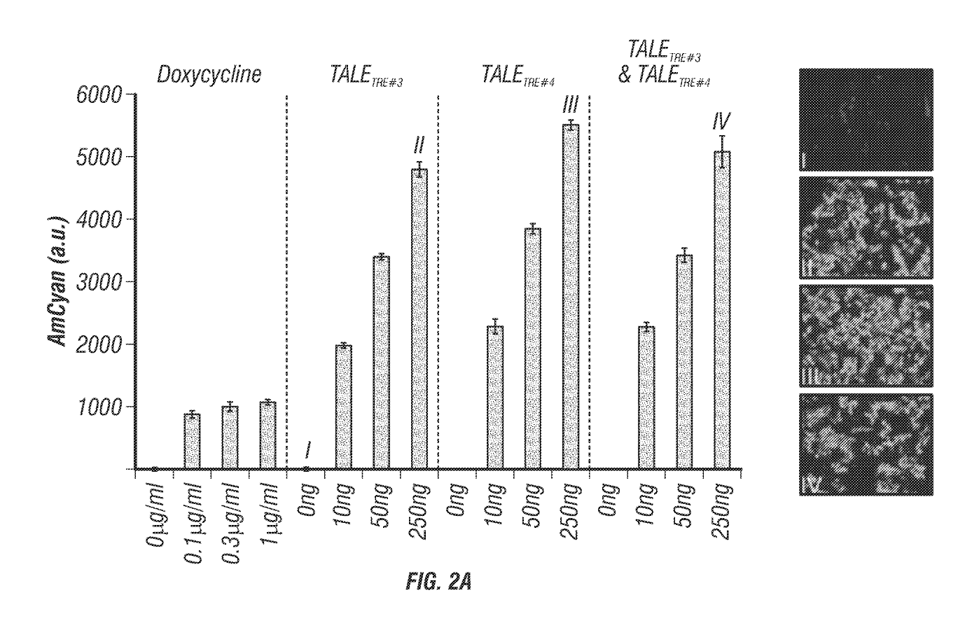

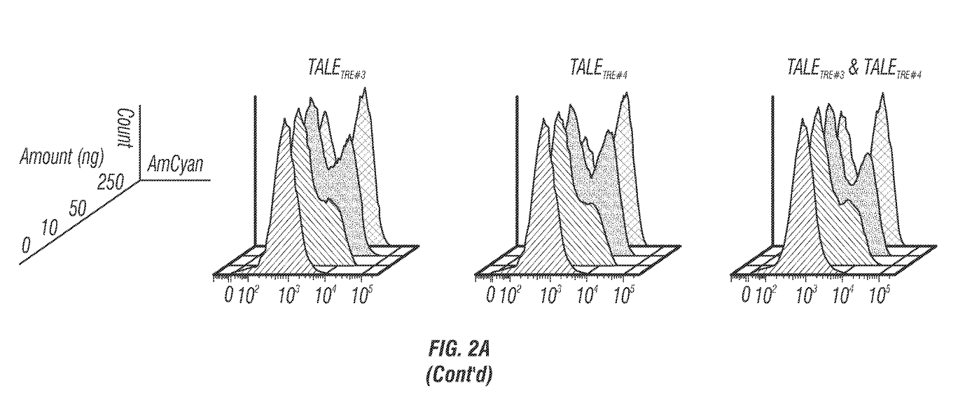

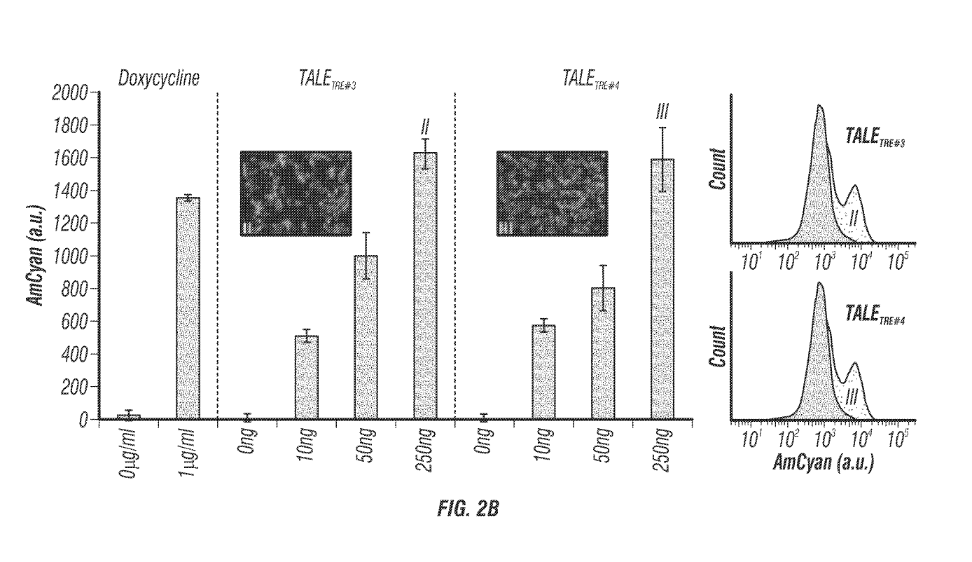

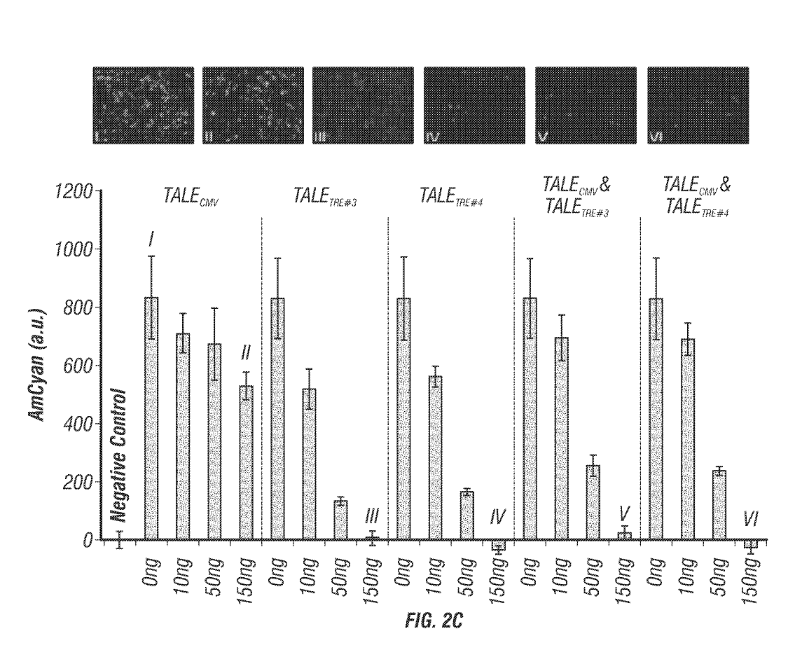

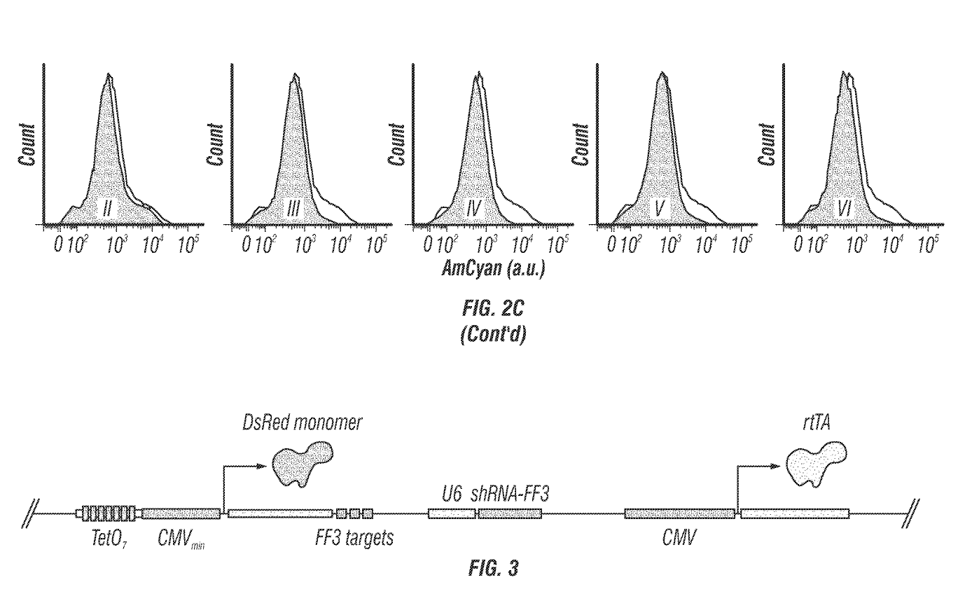

The inventors first explored the transactivation activities of TALE fusion proteins. In order to use a well-controlled environment, the inventors opted for the Flp-In system (Invitrogen) to generate a single-copy isogenic HEK293 stable cell line which contains an AmCyan fluorescent reporter gene under the control of a tetracycline responsive element (TRE) and minimum CMV promoter (FIG. 1c). The inventors note that the particular stably integrated gene cassette contains the reverse tetracycline-controlled transactivator (rtTA) protein transcript under the control of a CMV promoter, as well as other regulatory elements not relevant to this work (FIG. 3). In the presence of doxycycline, rtTA binds the TRE element and drives the expression of AmCyan (FIG. 2a).

The inventors then generated two TALEs, TALE_{TRE#3} and TALE_{TRE#4}, which bind to 7 repetitive sequences within the TRE elements (FIG. 1c and Table 1). Both TALEs were fused with a VP16 transactivation domain and transiently transfected into the stable cells. After 48 hours both TALE fusion proteins strongly induced the expression of AmCyan, as quantified by microscopy and flow cytometry (FIG. 2a). The results hold for 72 hours measurements. The transactivation activities of the TALE-VP16 proteins were significantly higher than doxycycline-induced rtTA. Notably, 10 ng of TALE_{TRE}-VP16 fusion protein (FIG. 2a, TALE_{TRE#3} and TALE_{TRE#4} panels) resulted in stronger AmCyan expression than the saturation concentration of doxycycline (1 μg/ml) (FIG. 2a). To investigate possible synergistic effects between the two TALE fusion proteins, equal amounts of both constructs were co-transfected, but no obvious such effects were observed (FIG. 2a). The inventors then sought to fuse the two TALEs with a weaker transactivation domain, the NF-kB p65 activation domain. The TALE_{TRE#3}-p65 and TALE_{TRE#4}-p65 fusions effectively induced the expression of AmCyan (FIG. 2b), as expected at lower levels (approximately 3-fold) compared to the VP16 fusions.

TABLE 1

| | | TAL | | |
|---|---|---|---|---|
| | | effector | | |
| Gene | | target | TAL | RVD |
| targets | Gene target sequences | sequences | effector | sequence |
| CMV | tagttattaatagtaatcaattacggggtcattagttcatagcccatatatggagttccgcg | CTATAT | TALE_{CMV} | HD NG NI |
| Prom | ttacataacttacggtaaatggcccgcctggctgaccgcccaacgacccccgcccattgac | AAGCA | | NG NI NG NI |
| oter | gtcaataatgacgtatgttcccatagtaacgccaatagggactttccattgacgtcaatgg | GAGCT | | NI NN HD NI |
| (TATA | gtggagtatttacggtaaactgcccacttggcagtacatcaagtgtatcatatgccaagta | | | NN NI NN |
| Box | cgccccctattgacgtcaatgacggtaaatggcccgcctggcattatgcccagtacatga | | | HD NG |
| Region) | ccttatgggactttcctacttggcagtacatctacgtattagtcatcgctattaccatggtg | | | |
| | atgcggttttggcagtacatcaatgggcgtggatagcggtttgactcacggggatttccaagt | | | |
| | ctccaccccattgacgtcaatgggagtttgattggcaccaaaatcaacgggactttccaa | | | |
| | aatgtcgtaacaactccgccccattgacgcaaatgggcggtaggcgtgtacggtggga | | | |
| | ggtctataagcagagctggtttagtgaaccgtcagatc* | | | |
| UbC | ggcctccgcgccgggttttggcgcctcccgcgggcgcccccctcctcacggcgagcg | ATATA | TALE_{Ubc} | NI NG NI NG |
| Prom | ctgccacgtcagacgaagggcgcaggagcgtcctgatccttccgcccggacgctcag | AGGAC | | NI NI NN NN |
| oter | gacagcggcccgctgctcataagactcggccttagaaccccagtatcagcagaaggac | GCGC | | NI HD NN |
| (TATA | attttaggacgggacttgggtgactctagggcactggttttctttccagagagcggaacag | | | HD NN HD |
| Box | gcgaggaaaagtagtcccttctcggcgattctgcggagggatctccgtggggcggtga | | | |
| Region) | acgccgatgattatataaggacgcgccgggtgtggcacagctagttccgtcgcagccg | | | |
| | ggatttgggtcgcggttatgalgtggatcgctgtgatcgtcacttggtgagtagcgggct | | | |
| | gctgggctggccggggctttcgtggccgccgggccgctcggtgggacggaagcgtgt | | | |
| | ggagagaccgccaagggctgtagtctgggtccgcgagcaaggttgccctgaactggg | | | |
| | ggttggggggagcgcagcaaaatggcggctgttcccgagtcttgaatggaagacgctt | | | |
| | gtgaggcgggctgtgaggtcgttgaaacaaggtggggggcatggtgggcggcaagaa | | | |
| | cccaaggtcttgaggccttcgctaatgcgggaaagctcttattcgggtgagatgggctgg | | | |
| | ggcaccatctggggaccctgacgtgaagtttgtcactgactggagaactcggtttgtcgt | | | |
| | ctgttgcgggggcggcagttatgcggtgccgttgggcagtgcacccgtacctttgggag | | | |
| | cgcgcgccctcgtcgtgtcgtgacgtcacccgttctgttggcttataatgcagggtgggg | | | |
| | ccacctgccggtaggtgtgcggtaggcttttctccgtcgcaggacgcagggttcgggcc | | | |
| | tagggtaggctctcctgaatcgacaggcgccggacctctggtgaggggagggataagt | | | |
| | gaggcgtcagtttctttggtcggttttatgtacctatcttcttaagtagctgaagctccggt | | | |
| | tttgaactatgcgctcggggttggcgagtgtgttttgtgaagttttttaggcaccttttgaa | | | |
| | atgtaatcatttgggtcaatatgtaattttcagtgttagactagtaaattgtccgctaaattc | | | |
| | tggccgtttttggcttttttgttagacga* | | | |
| TRE | cgagtttactccctatcagtgatagagaacgtatgtcgagtttactccctatcagtgataga | CCCTAT | TALE_{TRE#3} | HD HD HD |
| | gaacgatgtcgagtttactccctatcagtgatagagaacgtatgtcgagtttactccctatc | CAGTG | | NG NI NG |
| | agtgatagagaacgtatgtcgagtttactccctatcagtgatagagaacgtatgtcgagttt | AT | | HD NI NN |
| | atccctatcagtgatagagaacgtatgtcgagtttactccctatcagtgatagagaacgtat | | | NG NN NI |
| | gt | | | NG |
| TRE | cgagtttactccctatcagtgatagagaacgtatgtcgagtttactccctatcagtgataga | ATCAG | TALE_{TRE#4} | NI NG HD NI |
| | gaacgatgtcgagtttactccctatcagtgatagagaacgtatgtcgagtttactccctatc | TGATA | | NN NG NN |
| | agtgatagagaacgtatgtcgagtttactccctatcagtgatagagaacgtatgtcgagttt | GAGAA | | NI NG NI NN |
| | atccctatcagtgatagagaacgtatgtcgagtttactccctatcagtgatagagaacgtat | C | | NI NN NI NI |
| | gt | | | HD |

*TATA Box (Bold and Underlined)

Next, the inventors replaced the VP16 transactivation domain with a KRAB transcriptional repressor domain in the above TALE fusion proteins to determine their suppression effects. The expression of AmCyan was induced by 10 ng of TALE_{TRE#4}-VP16 (FIG. 2c) corresponding to the saturating doxycycline conditions. Different amounts of TALE_{TRE#3}-KRAB or TALE_{TRE#4}-KRAB were co-transfected and 72 hrs after transfection, both fusion proteins show strong suppression of the expression of AmCyan (FIG. 2c). 150 ng of either construct were able to abolish the expression of AmCyan. In fact, the TALE_{TRE#4}-KRAB at 150 ng suppressed AmCyan below its basal (due to TRE leakage) expression level (FIG. 2c, IV).

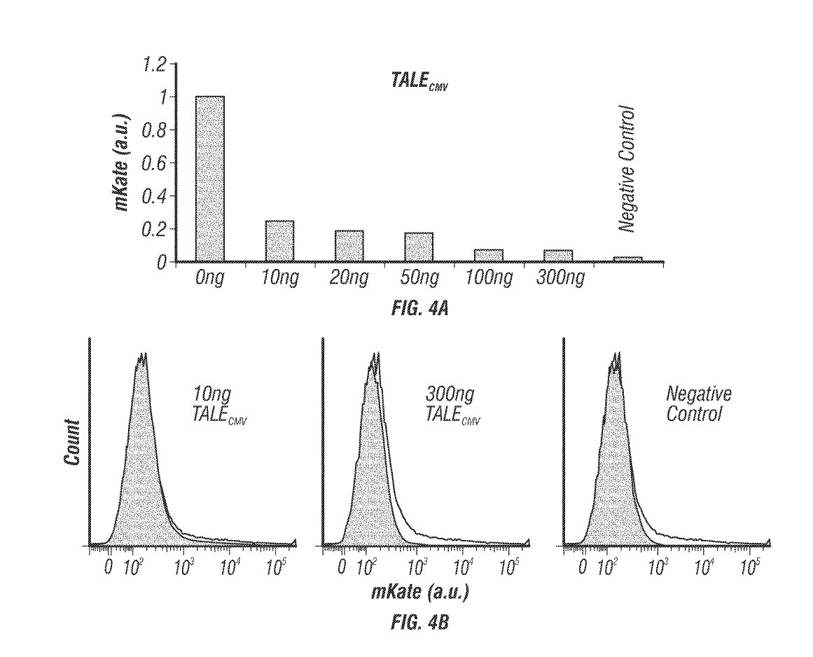

The inventors then generated a TALE that binds to the TATA box of the CMV promoter and fused it with KRAB domain (TALE_{CMV}-KRAB) (Table 1). The particular TALE_{CMV} has two binding sites within the inventors' stably integrated gene circuit: one within the CMV promoter (FIG. 3) and the other one within the CMVmin portion of the TRE/CMVmin promoter (FIG. 1). Compared to the aforementioned TALE_{TRE}-KRAB fusions, the suppression capacity of TALE_{CMV}-KRAB was significantly weaker. For instance, 50 ng of TALE_{TRE#3}-KRAB suppressed 84% of TALE_{TRE#4}-VP16-induced amCyan signal, and 50 ng of TALE_{TRE#4}-KRAB suppressed 80%. In comparison, the same amount of TALE_{CMV}-KRAB only suppressed 19% of the original fluorescent protein signal (FIG. 2c). To rule out of the possibility that the TALE_{CMV}-KRAB fusion does not efficiently bind to its target sequence, the inventors co-transfected this construct with 100 ng of CMV-mKate-PEST into plain HEK293 cells. Strong suppression of CMV-mKate-PEST was observed, as 100 ng of TALE_{CMV}-KRAB reduced its expression by 93% (FIG. 4). The inventors note that compared to amCyan, mKate fluorescent protein used in this experiment was fused with a PEST domain, which increases its turnover rate and may contribute to its higher susceptibility to TALE's suppression. Given that the TALE_{TRE}-KRAB and TALE_{CMV}-KRAB bind to different target sequences, the inventors explored their possible synergistic effects by co-transfecting equal amount of each construct. These combinations, tested at different total DNA levels, did not result in a greater suppression compared to the individual constructs (FIG. 2c).

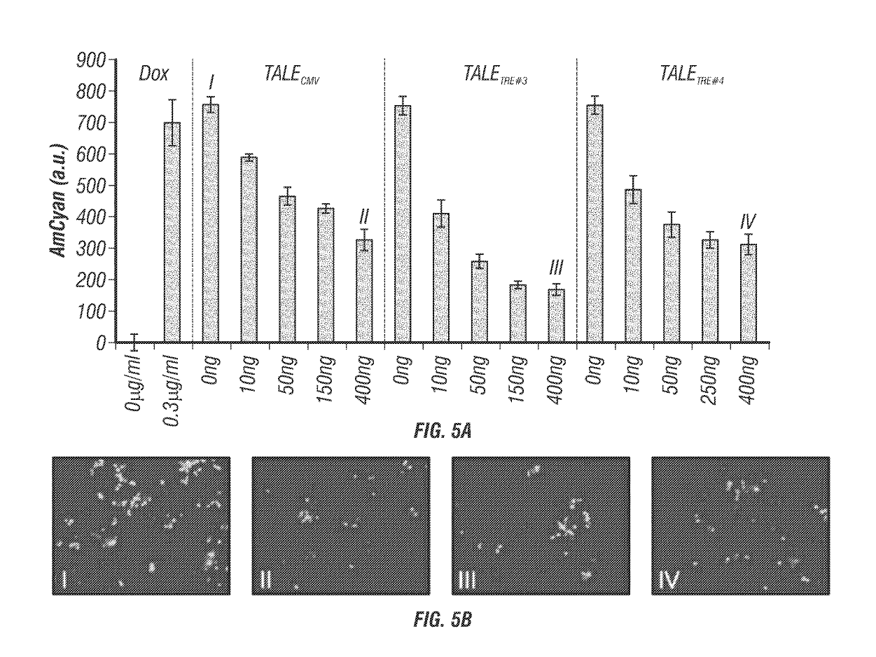

The inventors also tested whether the TALE-KRAB fusion proteins can suppress the induction effect of doxycycline. The stable cells were first transfected with different amounts of three TALE-KRAB constructs and then induced by 0.3 μg/ml doxycycline after 24 hrs. All three TALE-KRAB fusions still significantly suppressed the expression of AmCyan, though none were able to fully abolish the doxycycline-induced expression of the transgene (FIG. 5). The difference between the TALE_{TRE#4}-VP16 and doxycycline-induction experiments may be partially attributed to the transient transfection properties. When two plasmids are co-transfected (TALE_{TRE#4}-VP16 and TALE-KRAB) it is reasonable to assume higher probability for delivery into the same population of cells. In contrast, when inducing with doxycycline and transfecting TALE-KRAB there will be a subpopulation of induced cells that do not receive the TALE-KRAB plasmid and thus cannot be suppressed by TALE, increasing the population mean AmCyan level. To probe this effect the inventors transfected the cells with a constitutive YFP plasmid and induced the AmCyan using doxycycline; indeed, a significant portion of the AmCyan positive population failed to overlap with the YFP positive population.

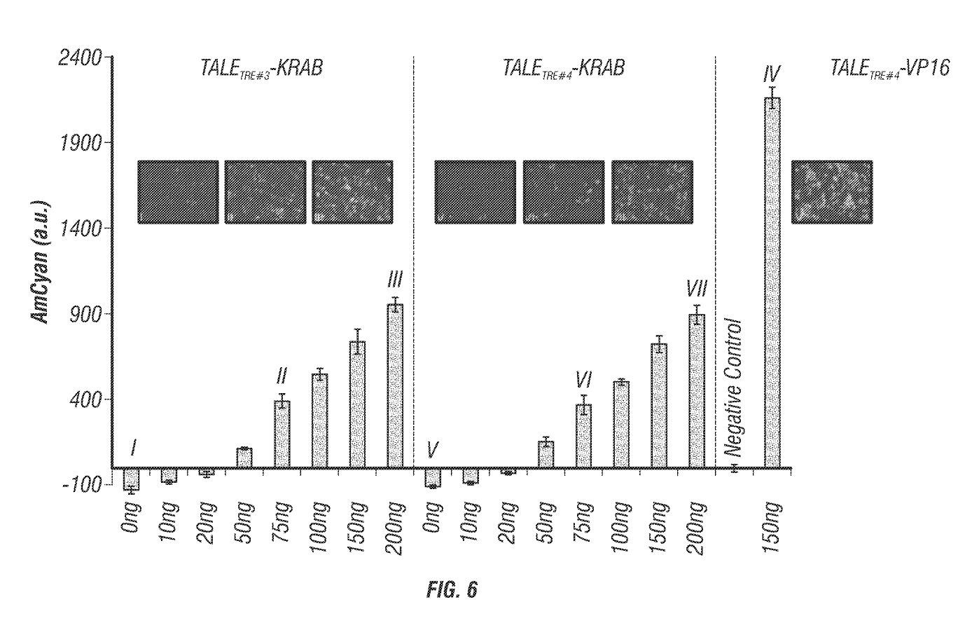

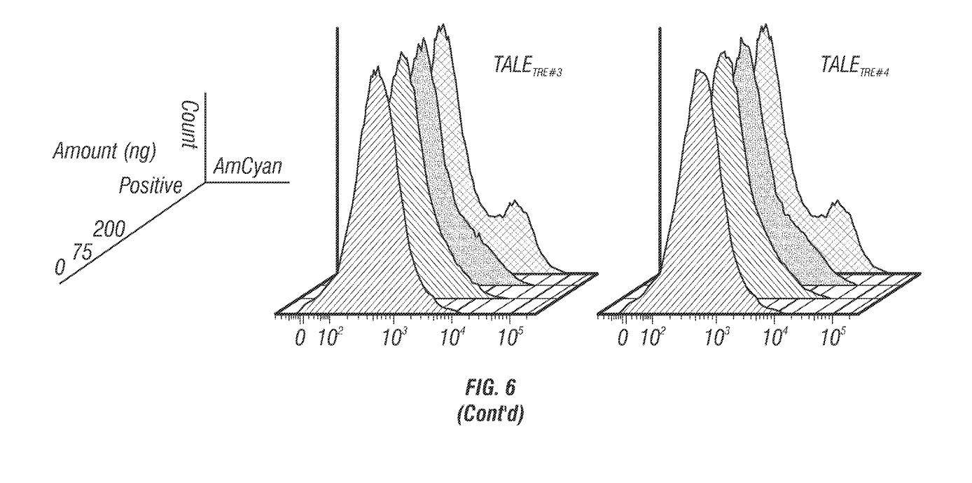

To conclude, the combination of TALE_{TRE}-KRAB and TALE_{TRE#4}-VP16 competing for overlapping or adjacent binding sites in the TRE element resulted in the most efficient repression of the transgene expression in the population of cells. The inventors note that adjusting the ratio between the two proteins can essentially reverse the inhibitory action. When the inventors co-transfected 150 ng of either TALE_{TRE}-KRAB constructs with different amount of TALE_{TRE#4}-VP16 into the stable cells, TALE_{TRE#4}-VP16 eventually counteracted the inhibitory effects of TALE_{TRE}-KRABs and induces the expression of AmCyan in a dose-dependent manner (FIG. 6).

TALE-Based Two-Hybrid System.

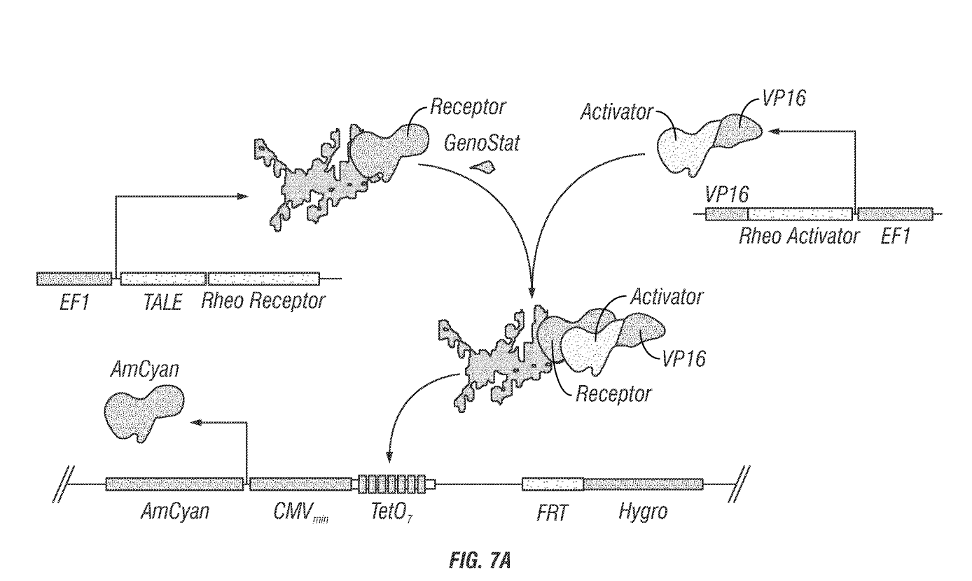

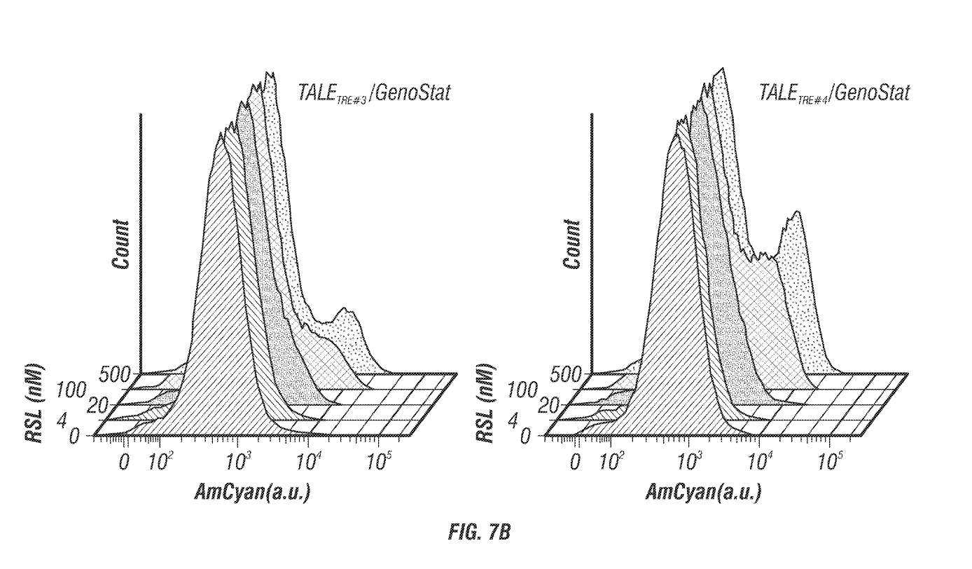

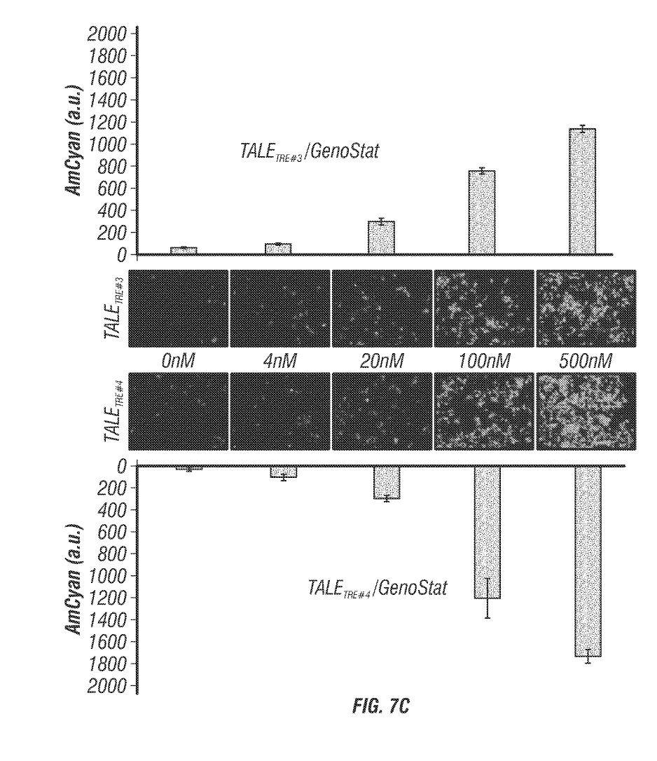

Regulating the function of TALE-fusions using small molecules was the inventors' next objective. The inventors observed that both TALE-VP16 and TALE-KRAB fusion constructs consist of two functional domains: first, the DNA-binding domain and, second, the transactivation or the repressor domain, which could potentially be separated into two components in a two-hybrid system (for reviews of mammalian two-hybrids, see Lievens et al., 2009; Lee and Lee, 2008). To test the feasibility of this TALE-based two-hybrid system, the inventors fused the TALE_{TRE#3} and TALE_{TRE#4} DNA binding domain with the Rheo Receptor (New England Biolabs). The Rheo Activator contains a VP16 transactivation domain but by itself lacks the capability of inducing expression of target genes. Upon induction with GenoStat ligand (Millipore), the Rheo Receptor and Rheo Activator form a heterodimer, which brings the VP16 domain to the proximity of TRE element and thus induces the expression of AmCyan. The inventors co-transfected the two plasmids into the stable cells and tested a range of different concentrations of GenoStat. 72 hours post transfection, the induction levels of AmCyan (FIG. 7) show a clear correlation with the GenoStat concentration, indicating that GenoStat specifically induced the association of TALE_{TRE}-Rheo Receptor and Rheo Activator, which resulted in a functional transactivation complex.

Interface of TALES with Endogenous Signals.

After the successful TALE-based control of chromosomal transgene expression and modulation of the activity of the TALEs using small molecules, the inventors' final objective was to interface a functional TALE to endogenous signals and consequently close the loop between cellular information and the transgene expression. The inventors first attempted to connect to an endogenous signaling pathway using a modification of the proposed TALE-based two-hybrid approach. The inventors selected to interface a TALE with the hypoxia pathway, given its general importance to cell health, but the approach should generally apply to any cellular heterodimerization reaction.

The central transcription factor of the hypoxia signaling, HIF-1 (hypoxia-inducible factor-1) is composed of two subunits: HIF-1α and ARNT (aryl hydrocarbon receptor nuclear translocator). ARNT is constitutively expressed, whereas HIF-1α is targeted to proteasome degradation under normoxia. Under hypoxia or CoCl₂ treatment, HIF-1α is stabilized and translocates to the nucleus, where it forms an active heterodimer with ARNT (Yuan et al., 2003).

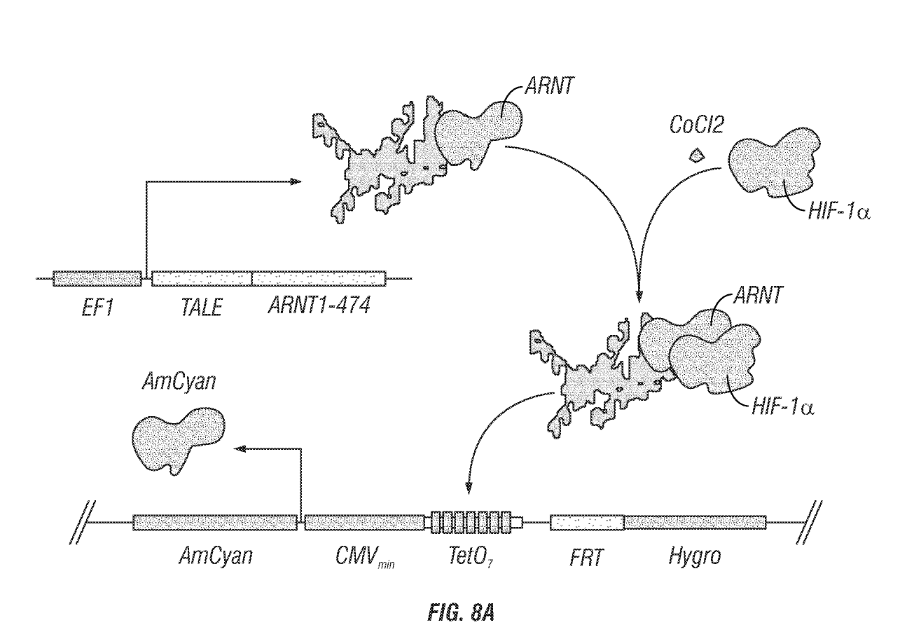

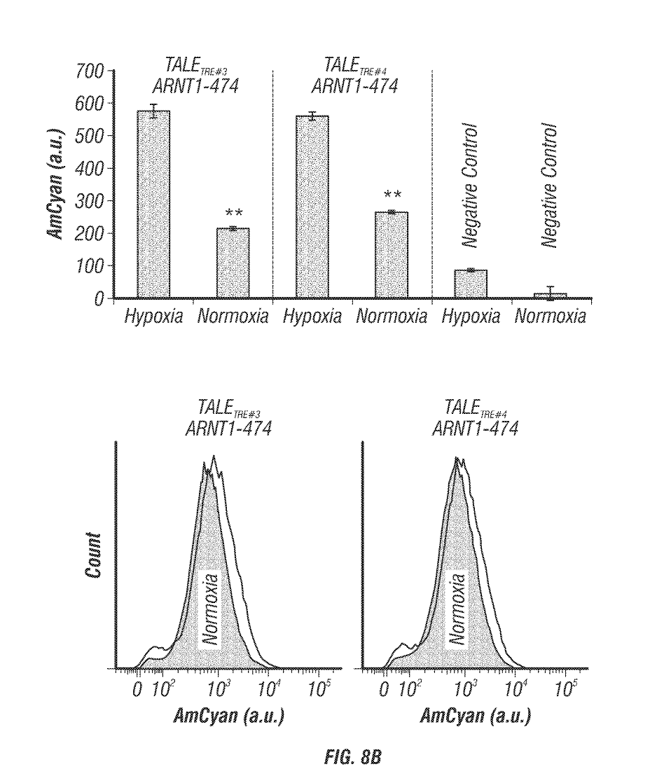

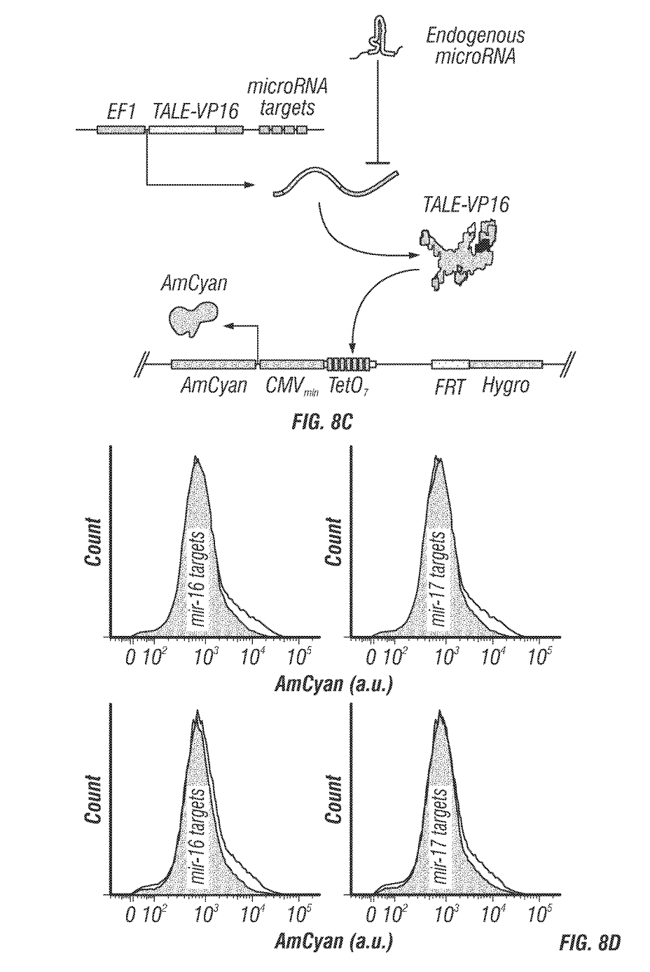

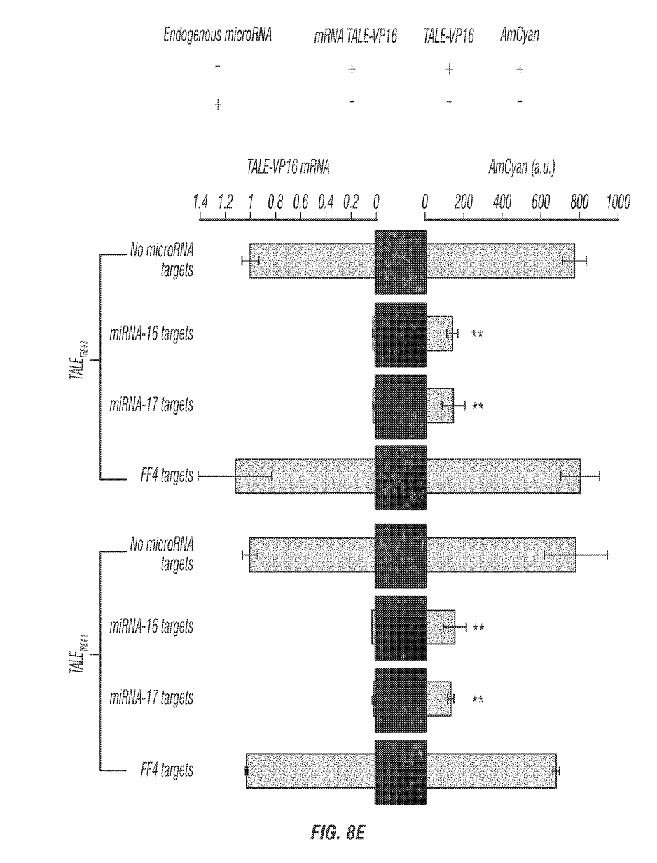

The inventors fused the amino acids 1-474 of human ARNT protein, which contain the HIF-1α-interacting domain bHLH-PAS but lack the transactivation domain, to the TALE_{TRE#3} and TALE_{TRE#4} DNA binding domains (FIG. 8a). The treatment of cells with CoCl2 (100 μM) significantly increased the expression of AmCyan fluorescent protein (FIG. 8b), indicating that the stabilized HIF-la protein formed a functional heterodimer with the TALE_{TRE}-ARNT1-474 fusions. In comparison, only minimal level of AmCyan expression was observed when the negative control, TALE_{TRE#4}-Rheo Receptor, was transfected. The inventors note that even without CoCl2 treatment (FIG. 8b), TALE_{TRE}-ARNT1-474 fusions induced an intermediate expression level of AmCyan. This result mostly likely arises from bHLH-PAS-dependent protein-protein cross-talk, as ARNT protein has been demonstrated to interact with other trans activators such as MOP1 and MOP2 (Long et al., 1999).

In addition to the hypoxia signaling, the inventors also selected microRNAs given their critical role in cells (Bartel, 2009) and on cell fate (Wijnhoven et al., 2007). To interface with endogenous microRNAs, the inventors invoke a method of microRNA-mediated repression that involves miRISC (microRNA and RISC complex) and direct endonucleolytic mRNA cleavage in a mechanism that highly resembles RNAi. Although rare in mammalian cells, it is known to occur when perfect complementarity between the microRNA target site and the miRISC group exists (Stegmeier et al., 2005; Xie et al., 2011).

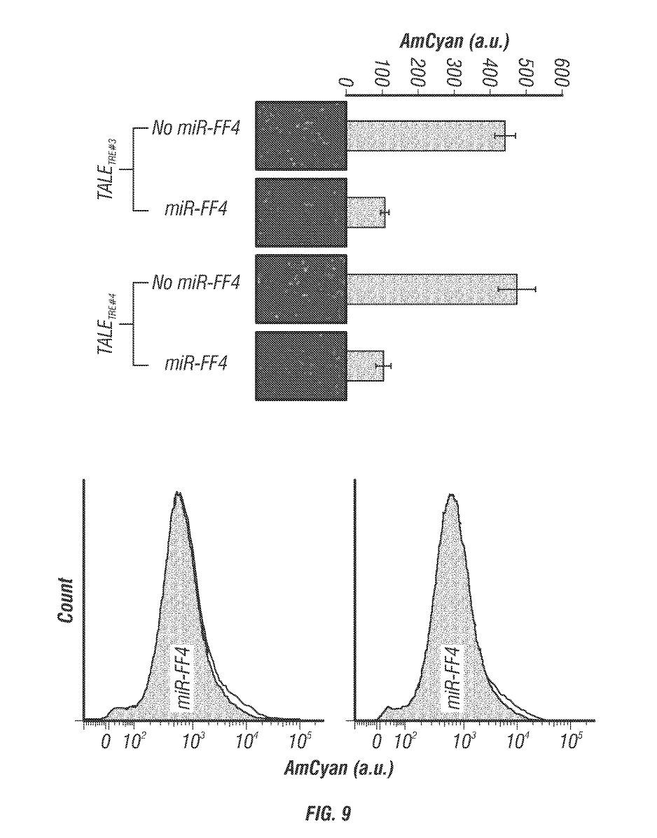

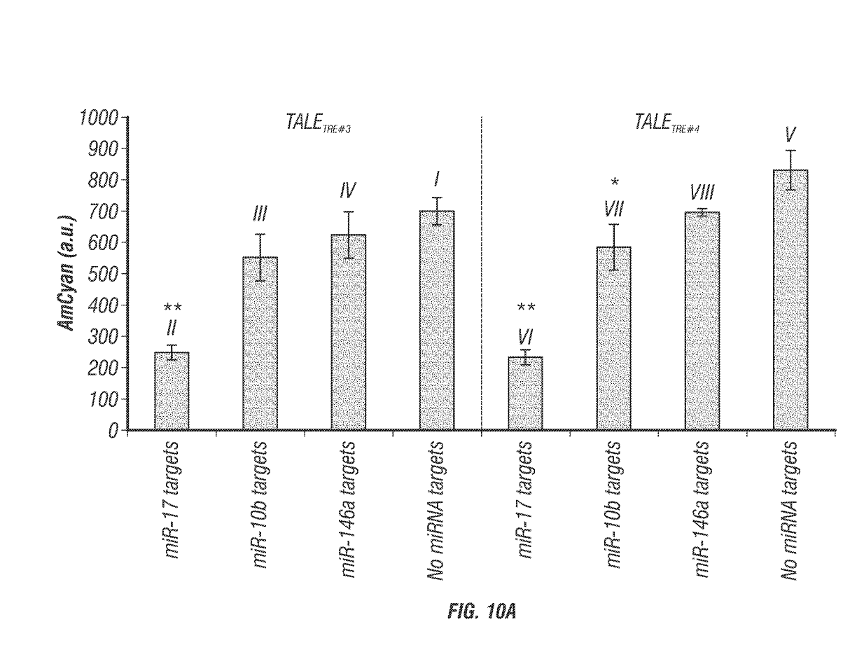

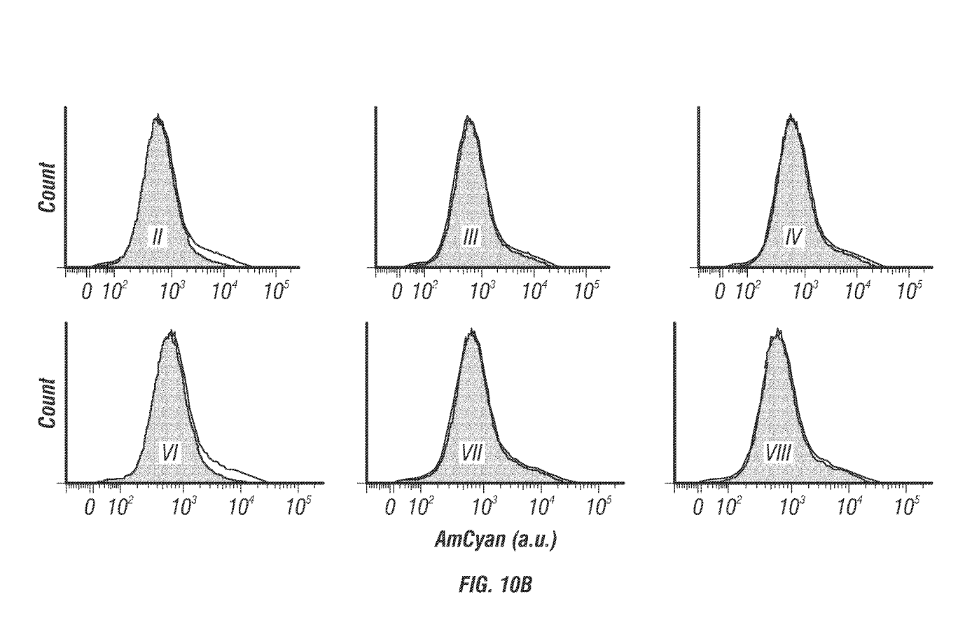

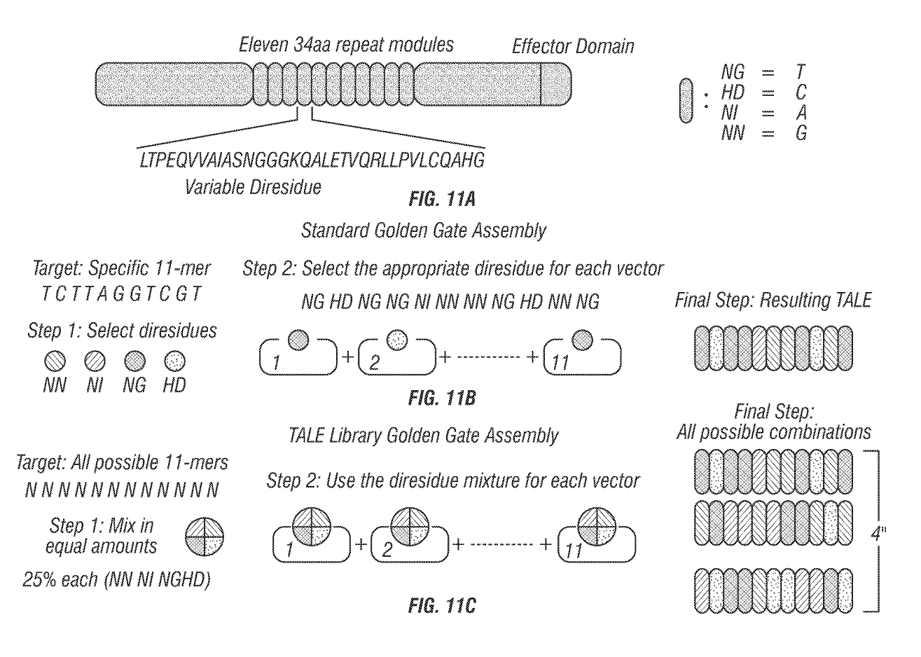

The inventors first focused on two of the most abundantly expressed miRNAs in HEK293 cells, miR-16 and miR-17, and incorporated 4 copies of their reverse complementary sequences into the 3′-UTR regions of TALE_{TRE#3}-VP16 and TALE_{TRE#4}-VP16 (FIG. 8c). A negative control construct was also generated by inserting 4 copies of the reverse complementary sequences of the artificial microRNA miR-FF4 (described in (Bleris et al., 2011; Rinaudo et al., 2007). Both miR-16 and miR-17 reduced the mRNA levels of TALE_{TRE}-VP16 by more than 95%, and effectively suppressed TALE_{TRE}-VP16's induction of AmCyan signals (FIG. 8d), while no down-regulation could be observed in the TALE_{TRE}-VP16 constructs which contain miR-FF4 targets (FIG. 8e). Note that the induction capacity of these constructs can be partially reduced by co-transfection of miR-FF4 (FIG. 9). In addition to the most abundant miRNAs (miR-16 and miR-17), the inventors further tested the suppression effects of miR-10b, which expression in HEK293 cells is at intermediate level, and miR-146a, which is absent in HEK293 cells. Four copies of the reverse complementary sequences of these two miRNAs were inserted into the inventors' TALE_{TRE}-VP16 constructs. Compared to miR-16 and miR-17, as expected, the suppression effects of miR-10b on TALE_{TRE}-VP16 were mild, resulting in as expected intermediate AmCyan activation (FIG. 10).

Discussion

The ability of signaling networks to detect, process, and react specifically to various signals is a key property of living cells and the implementation of systems (Holtz and Keasling, 2010; Ruder et al., 2011; Benenson, 2012) that reliably rewire such endogenous pathways can be a future therapeutic TALE-based application. The results presented here point to new generations of TALE hybrids and synthetic circuits engineered to detect and monitor endogenous signals with the capability of interfacing with biological pathways to apply predetermined and controllable action at the single-cell level.

The inventors show that competitive action of TALEs can be used to effectively control chromosomal gene expression. Furthermore, the inventors introduced a novel 2-hybrid system that can be used to regulate the activity of any TALE. Finally, as a proof of principle, the inventors demonstrated the successful interface of TALEs with hypoxia signaling and endogenous microRNA, essentially closing the loop by activating the stably integrated transgene cassette. In the future, TALE-based synthetic networks will be able to interface with the cellular environment to filter, amplify, and reliably transduce signals applying custom and fine-tuned control.

Methods

Recombinant DNA constructs. All TALE constructs were prepared using the Golden Gate TALEN and TAL effector kit (Addgene, catalog number: 1000000016) developed by Cermak et al. (2011). The TAL effector target sequences and their according RVD sequences were designed using the online tool TAL Effector Targeter (on the world wide web at boglabx.plp.iastate.edu/TALENT/). The target sequences are between 12-18 by and preceded by a T (Table 1). For the detailed cloning plan, see DNA constructs section and Table 2.

TABLE 2

| Primer | | |
|---|---|---|
| ID | Primer sequence (5′→3′) | Application |
| P1 | GTGCCACCTGGTCGACATCGATTATTGACTAGATC | forward primer for ClaI mutagenesis |
| P2 | GATCTAGTCAATAATCGATGTCGACCAGGTGGCAC | reverse primer for ClaI mutagenesis |
| P3 | CAGTACGGTACCCGGCCGCGACTCTAGATCATAATCA | forward primer for FF3X3-FF4X3 |
| P4 | CAGTACGCGGCCGCGATTATGATCAGTTATCTAGATC | reverse primer for FF3X3-FF4X3 |
| | CG | |
| P5 | CAGTACAGATCTTCTCACGGCTTCCCTCCCGAGGTGG | forward primer for PEST |
| P6 | CAGTACGTCGACTTAGACGTTGATCCTGGCGCTGGCG | reverse primer for PEST |
| P7 | CAGTACATCGATTAGTTATTAATAGTAATCAATTACG | forward primer for CMV-YFP-PEST |
| P8 | CAGTACATCGATGTTAAGATACATTGATGAGTTTGGA | reverse primer for CMV-YFP-PEST |
| | C | |
| P9 | CAGTACGGTACCGCGGGCCCGGGATCCACCGGATCTA | forward primer for removing FF3X3- |
| | | FF4X3 |
| P10 | CAGTACGCGGCCGCGTCGACTGCAGAATTCCTCACGA | reverse primer for removing FF3X3- |
| | CA | FF4X3 |
| P11 | CAGTACTCTAGAGAGCTCCACTTAGACGGCGAGGAC | forward primer for VP16 |
| | G | |
| P12 | CCAGTATCTAGACCCACCGTACTCGTCAATTCC | reverse primer for VP16 |
| P13 | CAGTACTCTAGACCAAAAAAGAAGAGAAAGGTCGAC | forward primer for KRAB |
| | G | |
| P14 | CCAGTATCTAGAAACTGATGATTTGATTTCAAATGC | reverse primer for KRAB |
| P15 | CCAGTATCTAGATTATTGGCCGCTGGAGCTGAT | forward primer for p65 |
| P16 | CCAGTATCTAGAATGGTGTTTCCTTCTGGGCAG | reverse primer for p65 |
| P17 | CTAGCTGGTACCCTCTAGATCATAATCAGCCTCGAGC | forward primer for miRtgt16X4 and |
| | | miRtgt17X4 |
| P18 | CTAGCTGCGGCCGCCAAGCTTATCGATCAAATGTGGT | reverse primer for miRtgt16X4 and |
| | ATG | miRtgt17X4 |
| P19 | CTAGCTGGTACCTGATCCTCTAGACCGCTTG | forward primer for FF4X3 |
| P20 | CTAGCTGCGGCCGCCGTGGACTCCAAGCTGGACA | reverse primer for FF4X3 |
| P21 | AGCTTCACAAATTCGGTTCTACAGGGTACACAAATTC | for miR-10b-tgtX4 |
| | GGTTCTAC | |
| P22 | TACCCTGTAGAACCGAATTTGTGTACCCTGTAGAACC | for miR-10b-tgtX4 |
| | GAATTTGTGA | |
| P23 | AGGGTACACAAATTCGGTTCTACAGGGTACACAAATT | for miR-10b-tgtX4 |
| | CGGTTCTACAGGGTAG | |
| P24 | GATCCTACCCTGTAGAACCGAATTTGTGTACCCTGTA | for miR-10b-tgtX4 |
| | GAACCGAATTTGTG | |
| P25 | AGCTTAACCCATGGAATTCAGTTCTCAAACCCATGGA | for miR-10b-tgtX4 |
| | ATTCAG | |
| P26 | TGAGAACTGAATTCCATGGGTTTGAGAACTGAATTCC | for miR-10b-tgtX4 |
| | ATGGGTTA | |
| P27 | TTCTCAAACCCATGGAATTCAGTTCTCAAACCCATGG | for miR-10b-tgtX4 |
| | AATTCAGTTCTCAG | |
| P28 | GATCCTGAGAACTGAATTCCATGGGTTTGAGAACTGA | for miR-10b-tgtX4 |
| | ATTCCATGGGTT | |
| P29 | CAGTACTCCGGATCTCACGGCTTCCCTCCCGAGGTGG | forward primer for PEST |
| P30 | CAGTACCTCGAGGATTATGATCTAGAGTCTTAGACGT | reverse primer for PEST |
| | TGATCCTGGCGCTGGCG | |
| P31 | CAGTACGGTACCGCGCCAGCGCCAGGATCAACGTC | forward primer for miR-10b-tgtX4 |
| P32 | CAGTACGCGGCCGCGATCAGTTATCTAGATCCGGTGG | reverse primer for miR-10b-tgtX4 |
| | ATCCT | |
| P33 | CTAGCTGGTACCTGATCCTCTAGACCGCTTG | forward primer for NotI mutagenesis |
| P34 | CTAGCTGCGGCCGCCGTGGACTCCAAGCTGGACA | reverse primer for NotI mutagenesis |
| P35 | CAGTACTCTAGAATGAAGCTACTGTCTTCTATCGAAC | forward primer for Rheo receptor |
| P36 | CAGTACTCTAGACTAGAGATTCGTGGGGGACTCGAGG | reverse primer for Rheo receptor |
| P37 | CAGTACTCCGGATCTCACGGCTTCCCTCCCGAGGTGG | forward primer for CMV-mKate- |
| | | PEST |
| P38 | CAGTACTCTAGATTAGACGTTGATCCTGGCGCTGGCG | reverse primer for CMV-mKate- |
| | | PEST |
| P39 | CAGTACTCTAGAATGGCGGCGACTACTGCCAACCCCG | forward primer for ARNT1-474 |
| P40 | CAGTACTCTAGACTATGTAGGCCGTGGTTCTTGGCTA | reverse primer for ARNT1-474 |

DNA Constructs.

EF1-FF3X3-FF4X3: EF1-GFP was purchased from Addgene (catalog number: 11154) (Matsuda and Cepko, 2004). A ClaI restriction site was generated in EF1-GFP by mutagenesis (QuickChange II Site-Directed Mutagenesis Kit, Genomics, catalog number: 200521) with primers P1 and P2. The FF3X3-FF4X3 sequence was PCR amplified from PBI-PCMV-DSRED-EXPRESS-ZSGREEN-FF3X3-FF4X3 using primers P3 and P4 and cloned into the above EF1 vector using KpnI and NotI sites. The FF3X3-FF4X3 sequence is 5′-AACGATATGGGCTGAATACAAAAACGATATGGGCTGAATACAAAAACGATATGG GCTGAATACAAACCGCTTGAAGTCTTTAATTAAACCGCTTGAAGTCTTTAATTAA ACCGCTTGAAGTCTTTAATTAAA-3′.

PCMV-YFP-PEST-EF1 and EF1: PCMV-YFP-C was purchased from Evrogen (catalog number: FP131). The PEST sequence was PCR amplified from Switchgear Genomics luciferase reporter system for SPERPINE1 (catalog number: 5721729) using primers P5 and P6 and cloned into PCMV-YFP-C vector using BglII/SalI sites. The PCMV-YFP-PEST was PCR amplified from above plasmid using primers P7 and P8 and cloned into EF1-FF3X3-FF4X3 vector using ClaI sites. To remove the FF3X3-FF4X3 sites, a cDNA sequence containing EF1 promoter was generated by using PCMV-YFP-PESTEF1-FF3X3-FF4X3 as the PCR template and primers P9 and P10. The PCR product and the template plasmid were digested with KpnI and NotI, ligated, and transformed to generate PCMV-YFP-PEST-EF1. This plasmid was subsequently digested with ClaI and self-ligated to further generate EF1 vector.

EF1-TALETRE#3-VP16 and EF1-TALETRE#4-VP16: The VP16-ER alpha plasmid was ordered from addgene (catalog number: 11351) (Chang et al., 1999). VP16 domain was PCR amplified from VP16-ER alpha using primers P11 and P12 and cloned into pTAL1 vector (Addgene, catalog number: 31031) using XbaI sites. pTAL1_{TRE#3}-VP16 and pTAL1_{TRE#4}-VP16 were prepared according to the instructions for the Golden Gate TALEN and TAL effector kit (Addgene). TALE_{TRE#3}-VP16 and TALE_{TRE#4}-VP16 were digested from pTAL1_{TRE#3}-VP16 and pTAL1_{TRE#4}-VP16 and cloned into EF1 vector using EcoRI sites.

EF1-TALE_{TRE#3}-KRAB, EF1-TALE_{TRE#4}-KRAB, and EF1-TALE_{CMV}-KRAB: KRAB domain was PCR amplified from PCMV-LacI-KRAB-FF3X3-FF4X3 using primers P13 and P14 and cloned into pTAL1 vector (Addgene) using XbaI sites. pTAL1_{TRE#3}-KRAB, pTAL1_{TRE#4}-KRAB, and pTAL1_{CMV}-KRAB were prepared according to the instructions for the Golden Gate TALEN and TAL effector kit (Addgene). TALE_{TRE#3}-KRAB, TALE_{TRE#4}-KRAB, and TALE_{CMV}-KRAB were digested from pTAL1_{TRE#3}-KRAB, pTAL1_{TRE#4}-KRAB, and pTAL1_{CMV}-KRAB respectively and cloned into EF1 vector using EcoRI sites.

EF1-TALE_{TRE#3}-p65 and EF1-TALE_{TRE#4}-p65: The pGyrB/puro plasmid was a gift from National Research Council of Canada through its Biotechnology Research Institute (Zhao et al., 2003). NF-Kb p65 domain was PCR amplified from the pGyrB/puro using primers P15 and P16. The PCR products and EF1-TALE_{TRE#3}-VP16 and EF1-TALE_{TRE#4}-VP16 were digested with XbaI, ligated and transformed to generate EF1-TALE_{TRE#3}-p65 and EF1-TALE_{TRE#4}-p65.

EF1-TALE_{CMV}-KRAB-FF3X3-FF4X3 and EF1-TALE_{UbC}-KRAB-FF3X3-FF4X3: pTAL1_{UbC}-KRAB was prepared according to the instructions for the Golden Gate TALEN and TAL effector kit (Addgene). TALE_{CMV}-KRAB and TALE_{UbC}-KRAB were digested from EF1-TALE_{CMV}-KRAB and pTAL1_{UbC}-KRAB respectively and cloned into EF1-FF3X3-FF4X3.

EF1-miR-16tgtX4, EF1-miR-17tgtX4, and EF1-FF4X3: miR-16tgtX4 and miR-17tgtX4 were PCR amplified from PCMV-ZSGREEN-miR16tgtX4 and PCMV-ZSGREEN-miR17tgtX4 using primers P17 and P18. The PCR products and EF1-FF3X3-FF4X3 were digested with KpnI and NotI, ligated, and transformed to generate EF1-miR-16tgtX4 and EF1-miR-17tgtX4. FF4X3 were PCR amplified from PTRE-TIGHT-BI-AMCYAN-DSRED-FF4X3 using primers P19 and P20. The PCR products and EF1-FF3X3-FF4X3 were digested with KpnI and NotI, ligated, and transformed to generate EF1-FF4X3. The miR16tgtX4 sequence is 5′-CGCCAATATTTACGTGCTGCTACGCCAATATTTACGTGCTGCTACGCCAATATTTA CGTGCTGCTACGCCAATATTTACGTGCTGCTA-3′. The miR17tgtX4 sequence is 5′-CTACCTGCACTGTAAGCACTTTGCTACCTGCACTGTAAGCACTTTGCTACCTGCAC TGTAAGCACTTTGCTACCTGCACTGTAAGCACTTTG-3′. The FF4X3 sequence is 5′-CCGCTTGAAGTCTTTAATTAAACCGCTTGAAGTCTTTAATTAAACCGCTTGAAGT CTTTAATTAAA-3′.

EF1-TALE_{TRE#3}-VP16-miR-16tgtX4, EF1-TALE_{TRE#3}-VP16-miR-17tgtX4, EF1-TALE_{TRE#3}-VP16-miR-FF4X3, EF1-TALE_{TRE#4}-VP16-miR-16tgtX4, EF1-TALE_{TRE#4}-VP16-miR-17tgtX4, and EF1-TALE_{TRE#4}-VP16-miR-FF4X3: TALE_{TRE#3}-VP16 and TALE_{TRE#4}-VP16 were digested from pTAL1_{TRE#3}-VP16 and pTAL1_{TRE#4}-VP16 and cloned into EF1-miR-16tgtX4, EF1-miR-17tgtX4 and EF1-FF4X3 to generate above plasmids with respective microRNA targets.

miR-10b-tgtX4 and miR-146a-tgtX4: For miR-10b-tgtX4, equalmolar (10 μM final concentration) P21 and P22, or P23 and P24 were mixed in 1XT4 Polynucleotide kinase buffer (total volume 20 μL, New England Biolabs, catalog number: M0201), heated to 95° C. and slowly cooled down by 1° C./min to 25° C. on a PCR block. ATP (final concentration 0.5 mM, New England Biolabs, catalog number: P0756) and T4 Polynucleotide kinase (final concentration 0.5 units/μL, New England Biolabs, catalog number: M0201) were then added and the reaction was kept at 37° C. for 1 hr. 2 μL of P21:P22 and 2 μL P23:P24 were mixed in 1×T4 DNA ligase buffer (New England Biolabs, catalog number: M0202) with T4 DNA ligase (final concentration 0.5 units/μL, New England Biolabs, catalog number: M0202) at room temperature for 1 hr. The miR-10bX4 product was resolved by and purified from 4% Metaphor agarose gel (Lonza, catalog number: 50181). For miR-146a-tgtX4, primers P25, P26, P27 and P28 were used and the procedures were essentially identical. The miR-10b-tgtX4 sequence is 5′-CACAAATTCGGTTCTACAGGGTACACAAATTCGGTTCTACAGGGTACACAAATTC GGTTCTA CAGGGTACACAAATTCGGTTCTACAGGGTA-3′. The miR-146a-tgtX4 sequences is 5′-AACCCATGGAATTCAGTTCTCAAACCCATGGAATTCAGTTCTCAAACCCATGGAA TTCAGTTCTCAAACCCATGGAATTCAGTTCTCA-3′.

CMV-YFP-PEST-miR-10b-tgtX4 and CMV-YFP-PEST-miR-146a-tgtX4: CMV-YFP-C was purchased from Evrogen. The PEST sequence was PCR amplified from Switchgear Genomics luciferase reporter system for SPERPINE1 (catalog number: 5721729) using primers P29 and P30 and cloned into CMV-YFP-C vector using BspeI/XhoI sites. The above miR-10b-tgtX4 and miR-146a-tgtX4 inserts were then cloned into CMV-YFP-PEST using BamHI and HindIII sites to generate CMV-YFP-PEST-miR-10b-tgtX4 and CMV-YFP-PEST-miR-146a-tgtX4.

EF1-TALE_{TRE#3}-VP16-miR-10b-tgtX4 and EF1-TALE_{TRE#4}-VP16-miR-10b-tgtX4: The miR-10btgtX4 was PCR amplified from CMV-YFP-PEST-miR-10b-tgtX4 using primers P31 and P32. The PCR products and EF1-FF3X3-FF4X3 were digested with KpnI and NotI, ligated, and transformed to generate EF1-miR-10b-tgtX4. TALE_{TRE#3}-VP16 and TALE_{TRE#4}-VP16 were digested from pTAL1_{TRE#3}-VP16 and pTAL1_{TRE#4}-VP16 and cloned into EF1-miR-10b-tgtX4 to generate EF1-TALE_{TRE#3}-VP16-miR-10btgtX4 and EF1-TALE_{TRE#4}-VP16-miR-10b-tgtX4.

EF1-TALE_{TRE#3}-VP16-miR-146a-tgtX4 and EF1-TALE_{TRE#4}-VP16-miR-146a-tgtX4: The NotI sites within the ORFs of EF1-TALE_{TRE#3}-VP16-miR-16tgtX4 and EF1-TALE_{TRE#4}-VP16-miR-16tgtX4 were mutated by mutagenesis (QuickChange II Site-Directed Mutagenesis Kit, Genomics) with primers P33 and P34. The miR-146a-tgtX4 was PCR amplified from CMV-YFP-PEST-miR-146a-tgtX4 using primers P31 and P32. The PCR products and mutated EF1-TALE_{TRE#3}-VP16-miR-16tgtX4 or EF1-TALE_{TRE#4}-VP16-miR-16tgtX4 were digested with KpnI and NotI, ligated, and transformed to generate EF1-TALE_{TRE#3}-VP16-miR-146a-tgtX4 and EF1-TALE_{TRE#4}-VP16-miR-146a-tgtX4.

EF1-TALE_{TRE#3}-RheoReceptor and EF1-TALE_{TRE#4}-RheoReceptor: The RheoSwitch Mammalian Inducible Expression System was purchased from New England Biolabs (catalog number: E3000). The RheoReceptor ORF was PCR amplified using P35 and P36. The PCR products and EF1-TALE_{TRE#3}-VP16 or EF1-TALE_{TRE#4}-VP16 were digested with XbaI, ligated, and transformed to generate EF1-TALE_{TRE#3}-RheoReceptor and EF1-TALE_{TRE#4}-RheoReceptor.

EF1-RheoActivator: The RheoActivator ORF was digested from FF4-YFP-TRE-BI-RheoActivator-FF4 with KpnI and NotI. EF1-FF3X3-FF4X3 was digested with same restriction enzymes and ligated with RheoActivator ORF to generate EF1-RheoActivator.

CMV-mKate-PEST: The CMV-mKate-C plasmid was first transformed into dam⁻/dcm⁻ competent E. coli (New England Biolabs, catalog number: C2925) to free the XbaI restriction site. The PEST sequence was PCR amplified from Switchgear Genomics luciferase reporter system for SPERPINE1 (catalog number: 5721729) using primers P37 and P38 and cloned into CMV-mKate-C vector using BspeI/XbaI sites.

EF1-TALE_{TRE#3}-ARNT1-474 and EF1-TALE_{TRE#4}-ARNT1-474: Total mRNA was harvested from HEK293 cells using RNeasy Mini Kit (Qiagen, catalog number: 74104). The first-strand cDNA was synthesized using QuantiTech Rev. Transcription Kit (Qiagen, catalog number: 205310). The amino acids 1-474 of human ARNT was then cloned using primers P39 and P40. This cDNA was then cloned into EF1-TALE_{TRE#3} and EF1-TALE_{TRE#4} using XbaI site.

Cell Culture and Transient Transfection.

A HEK293 stable cell line that harbors the Tetracycline Responsive Element (TRE) AmCyan transcript was generated using Flp-In System (Invitrogen, catalog number: K6010-01) according to the manufacturer's instructions. The cells were maintained at 37° C., 100% humidity and 5% CO, The cells were grown in Dulbecco's modified Eagle's medium (DMEM, Invitrogen, catalog number: 11965-1181) supplemented with 10% Fetal Bovine Serum (FBS, Invitrogen, catalog number: 26140), 0.1 mM MEM non-essential amino acids (Invitrogen, catalog number: 11140-050), 0.045 units/mL of Penicillin and 0.045 units/mL of Streptomycin (Penicillin-Streptomycin liquid, Invitrogen, catalog number: 15140), and 50 μg Hygromycin B (Invitrogen, catalog number: 10687-010). To pass the cells, the adherent culture was first washed with PBS (Dulbecco's Phosphate Buffered Saline, Mediatech, catalog number: 21-030-CM), then trypsinized with Trypsin-EDTA (0.25% Trypsin with EDTAX4Na, Invitrogen, catalog number: 25200) and finally diluted in a fresh medium upon reaching 50-90% confluence. To maintain plain HEK293 cells, the procedures were essentially the same, except that no Hygromycin B was included in the growth medium.

For transient transfections, ˜300 thousand cells in 1 mL of complete medium were plated into each well of 12-well culture treated plastic plates (Griener Bio-One, catalog number: 665180) and grown for 16-20 hours. For Lipofectamine LTX transfection, up to 1 μg of the plasmid was added to 200 μL of DMEM and 2 μL Lipofectamine LTX (Invitrogen, catalog number: 94756). Transfection solutions were mixed and incubated at room temperature for 30 minutes. The transfection mixture was then applied to the cells and mixed with the medium by gentle shaking. When applicable, doxycycline (Clontech, catalog number: 631311) was added three hours after transfection.

Fluorescence Microscopy.

All microscopy was performed 48-72 hours post transfection. The live cells were grown on 12-well plates (Greiner Bio-One) in the complete medium. Cells were imaged using the Olympus IX81 microscope and a Precision Control environmental chamber. The images were captured using a Hamamatsu ORCA-03 Cooled monochrome digital camera. The filter sets (Chroma) are as follows: ET436/20x (excitation) and ET480/40 m (emission) for AmCyan, ET560/40x (excitation) and ET630/75 m (emission) for mKate, ET500/20x (excitation) and ET535/30 m (emission) for YFP (Yellow Fluorescent Protein). Data collection and processing was performed in software package Slidebook 5.0. All images within a given experimental set were collected with the same exposure times and underwent identical processing.

Flow Cytometry.

48-72 hours post transfection cells from each well of the 12-well plates were trypsinized with 0.1 mL 0.25% Trypsin-EDTA at 37° C. for 3 mins Trypsin-EDTA was then neutralized by adding 0.9 mL of complete medium. The cell suspension was centrifuged at 1000 rpm for 5 mins and after removal of supernatants, the cell pellets were resuspended in 0.5 mL PBS buffer. The cells were analyzed on a BD LSRFortessa flow analyzer. AmCyan was measured with a 445-nm laser and a 515/20 band-pass filter, mKate with a 561-nm laser, 610 emission filter and 610/20 band-pass filter, and YFP with a 488-nm laser, a 535 emission filter and 545/35 band-pass filter.

For experiments performed in TRE_AmCyan HEK293 cells, 100,000 events were collected. A FSC (forward scatter)/SSC (side scatter) gate was generated using a un-transfected negative sample and applied to all cell samples. The mean values of AmCyan reporter fluorescence were then collected and processed by FlowJo. The average of the means of AmCyan from three control samples which were transfected with empty plasmids (EF1-FF3X3-FF4X3, see DNA constructs section) were set as baseline values and were subtracted from all other experimental samples. All experiments were performed in triplicates.

For experiments performed in plain HEK293 cells, 50,000 events were collected. A FSC (forward scatter)/SSC (side scatter) gate was first generated using a un-transfected negative sample and applied to all cell samples. The cells were further gated to select the YFP+ populations. The mean values of mKate and YFP were collected and processed by FlowJo. The ratios of mKate/YFP were then calculated.

Quantitative Reverse Transcription-PCR.

48 hours post transfection, total RNA was extracted from TRE_AmCyan HEK293 cells using an RNeasy Mini kit (Qiagen, catalog number: 74104) following the manufacturer's protocol. First-strand synthesis was performed using QuantiTect Reverse Transcription kit (Qiagen, catalog number: 205311). Quantitative PCR was performed using KAPA SYBR FAST Universal qPCR kit (KAPA Biosystems, catalog number: KK4601). Glyceraldehyde-3-phosphate dehydrogenase (GAPDH) sequences were used for normalization. The forward primer for GAPDH was 5′-AATCCCATCACCATCTTCCA-3′, and the reverse primer for GAPDH was 5′-TGGACTCCACGACGTACTCA-3′. The forward primer for TALE was 5′-CTCCACTTAGACGGCGAGGA-3′, and the reverse primer for TALE was 5′-GAAGTCGGCCGTATCCAGAG-3′. The thermal cycling conditions were 3 min at 95° C. followed by 40 cycles of denaturation for 15 s at 95° C. and annealing for 30 s at 60° C. Normalized data were used to compare relative levels of TALE-VP16 transcripts which contained different miRNA targets using ΔΔCt analysis.

Statistical Analysis.

The values of AmCyan reporter fluorescence are reported as mean with standard deviation. The significance values between sample groups were calculated by the Student's ‘t’-test, and P-values less than 0.05 were taken as significance in all the experiments.

### Example 2

### Construction of a 11-Mer TALE-VP16 Library

The current research paradigm relies on designing TALEs for defined DNA targets based on TALE-DNA binding algorithms such as TALE-NT 2.0 (TALE Effector Nucleotide Targeter 2.0). One potential limitation of this approach lies in the fact that the available algorithms may not yet capture reliably the TALE-DNA target interactions. Indeed some of the inventors' designed TALEs for above TRE-minimum CMV promoter failed to elicit desirable binding affinities. Therefore, the inventors wanted to establish a different alternative by constructing TALE libraries which consist of all possible combinations of tandem repeats and subject this library to properly designed screening assay in hope of capturing the strongest binding events.

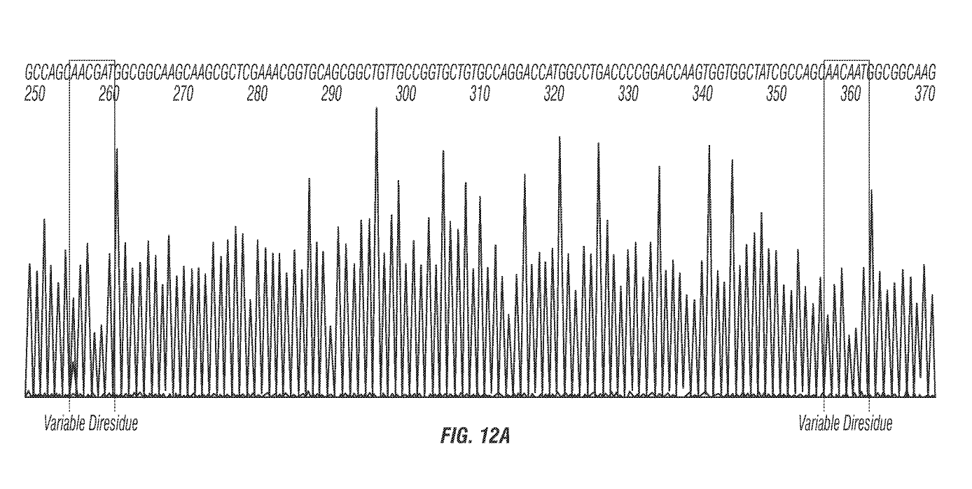

To construct a 11-mer TALE-VP16 library, the inventors developed a new protocol based on the Golden Gate assembly (FIG. 11b). The inventors introduce a mixture of equal amount of all four possible building modules in the reaction. For example, as illustrated in FIG. 11c, for position 1, 25 ng of each of NN1, NI1, NG1 and HD1 can be included. Since all four modules only differ at RVDs (positions 12 and 13), and the flanking sequences for BasI-based digestion/T4 DNA ligase-based ligation reactions remain identical, these four modules will have an equal probability in getting incorporated into the final TALE construct. The inventors were able to first separately prepare the pFUS_A library which contains all possible combinations of 10 tandem repeats and pFUS_B library which contains one repeat. These two component libraries were then conjoined to make the final 11-mer TALE library which covers all possible 11-mer DNA targets (4¹¹=4,194,304).

### Example 3

### Library Quality

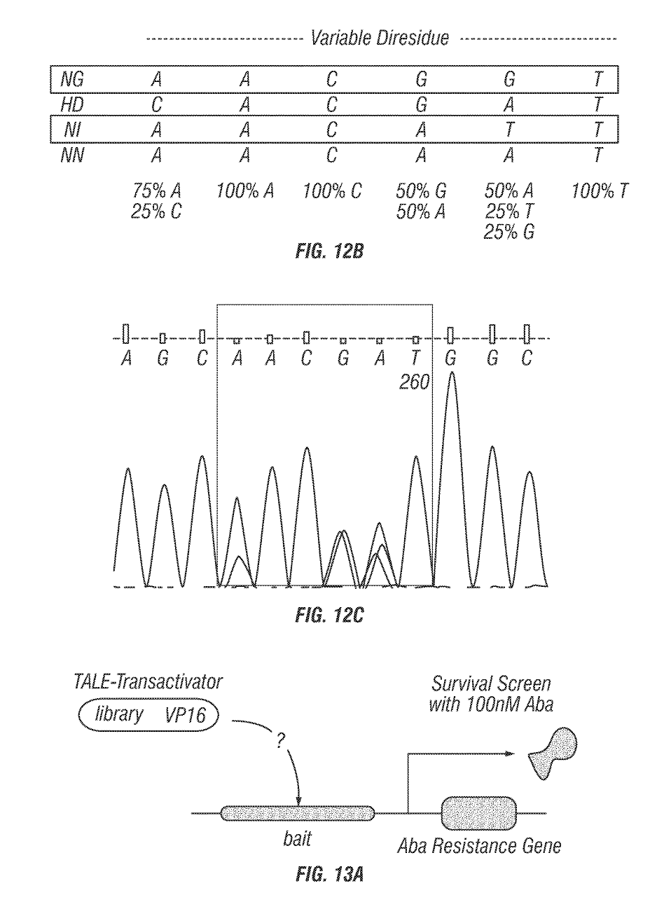

To test the library quality the inventors subjected their TALE-VP16 library to standard Sanger sequencing using primers flanking the TALE DNA binding domain. The inventors noted that (a) there are 6-nucleotide long repeats, spaced by 102 nucleotides, which showed “noisy” signals (FIG. 12a). This phenomenon matches the fact that each TALE tandem repeat contains 102 nucleotides and its RVDs are 6-nucleotide in length; and more importantly (b) the composition of different peaks within these 6-nucleotide elements closely tracks the prediction (FIG. 12b) when equal amount of all four possible RVDs are mixed. For example, at position 4 of the RVD sequence, nucleotides A and G are predicted to each contribute 50% of the occurrence, which was observed in the inventors' sequencing results (FIG. 12c).

### Example 4

### Positive Screening in Yeast Cells

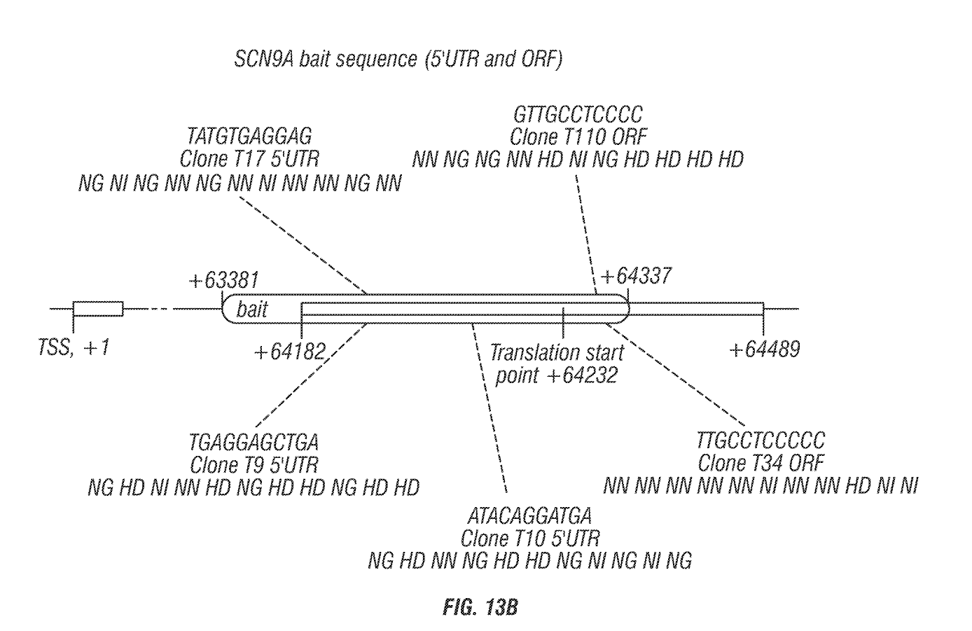

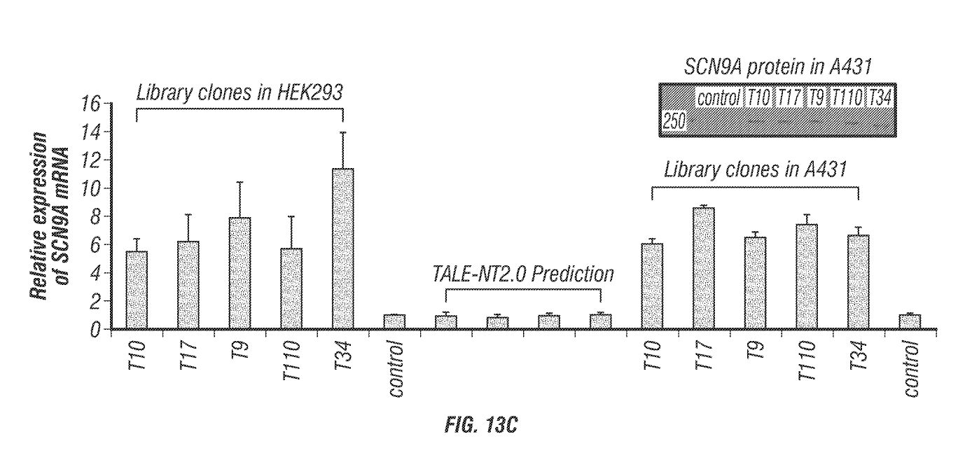

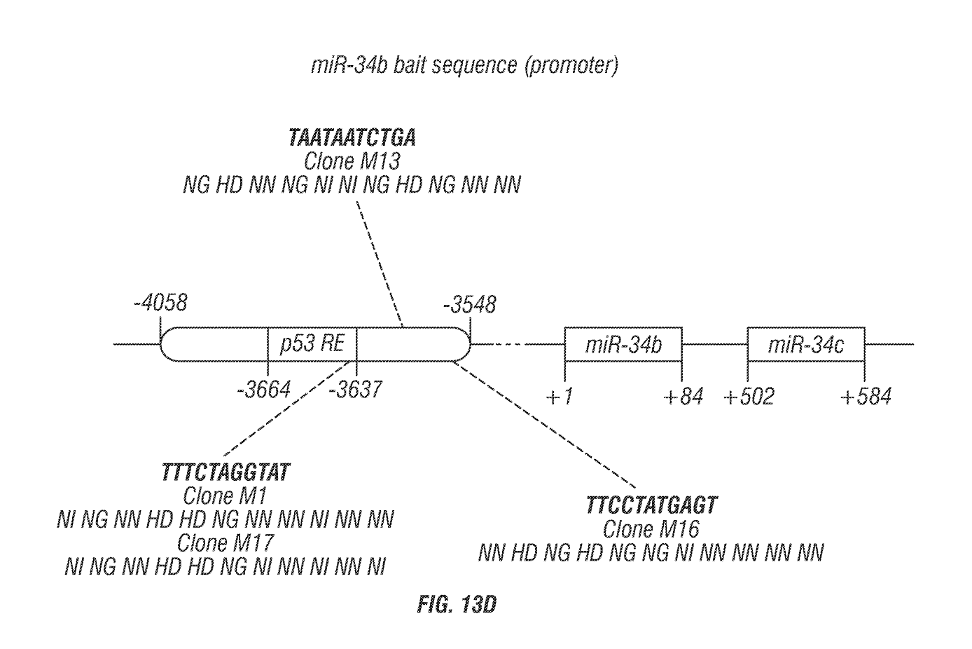

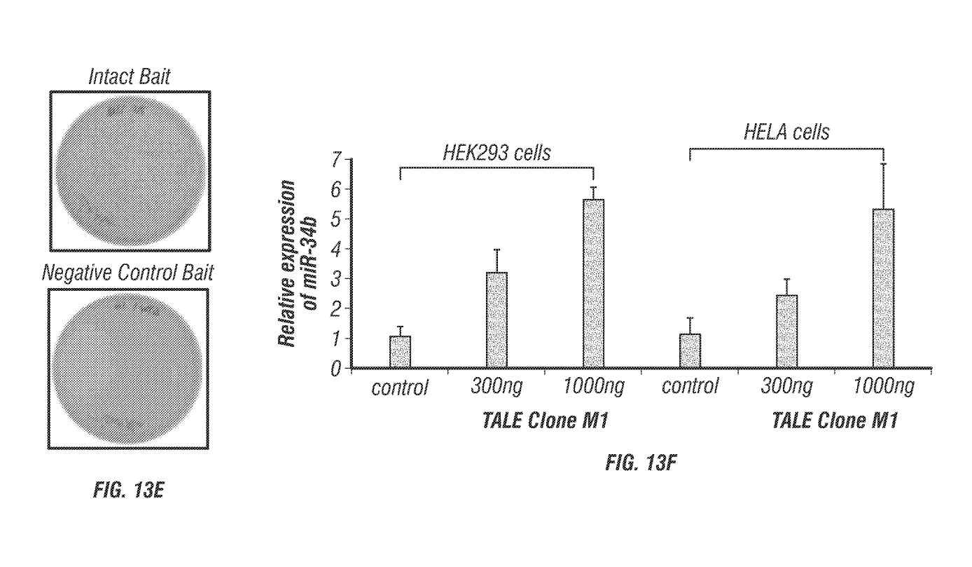

The inventors tested the functional integrity of the TALE-VP16 library using a yeast one-hybrid assay (Matchmaker Gold Yeast One-Hybrid Library Screening System, Clontech). In this assay, the inventors cloned part of the 5′-UTR and the ORF of human SCN9A gene in front of an antibiotic resistance gene (Aureobasidin A resistance gene) in yeast (bait), and then applied the library (prey) screened up to 1 million individual clones (FIG. 13a). The positive clones were confirmed by re-streaking on Aureobasidin A-containing agar plates (FIG. 13b). The TALE-VP16 expression plasmids were then rescued and sequenced to extract the RVD sequences (FIG. 13c). Using TALE-NT 2.0 tool, the inventors were able to determine that these 13 positive clones are predicted to bind to either the plus or minus strand of three specific locations within the SCN9A bait sequence.

The inventors applied two methods to confirm these observations. First, the inventors generated baits which exclude those predicted DNA target sites. While the isolated TALE-VP16 fusions could induce expression of Aureobasidin A resistance gene when the bait sequence was intact (FIG. 13d, left), it failed to do so when the DNA target site was removed (FIG. 13d, right). Secondly, the inventors cloned these TALE-VP16 fusions into a mammalian expression vector and after transiently transfected them into HEK293 cells, measured the expression levels of SCN9A mRNA by quantitative RT-PCR. All fusions were able to effectively drive the overexpression of SCN9A gene (up to 11-fold increase) (FIG. 13e). From above results, the inventors noticed that, which has not yet been reported, TALE-VP 16 can be designed to bind to the minus strand of a target sequence and in addition, can also be designed to target sequences within ORF. It is interesting to note that, after replacing the VP16 domain with a KRAB suppressor domain, the fusions failed to down-regulate SCN9A expression. One possible explanation to this difference is that the transactivator function of VP16 happens at both initiation and elongation steps during transcription, while the suppression effects of KRAB most probably only at initiation. Therefore, for TALE-KRAB fusions, the target sequences before TSS (transcriptional start site) should be chosen as baits.

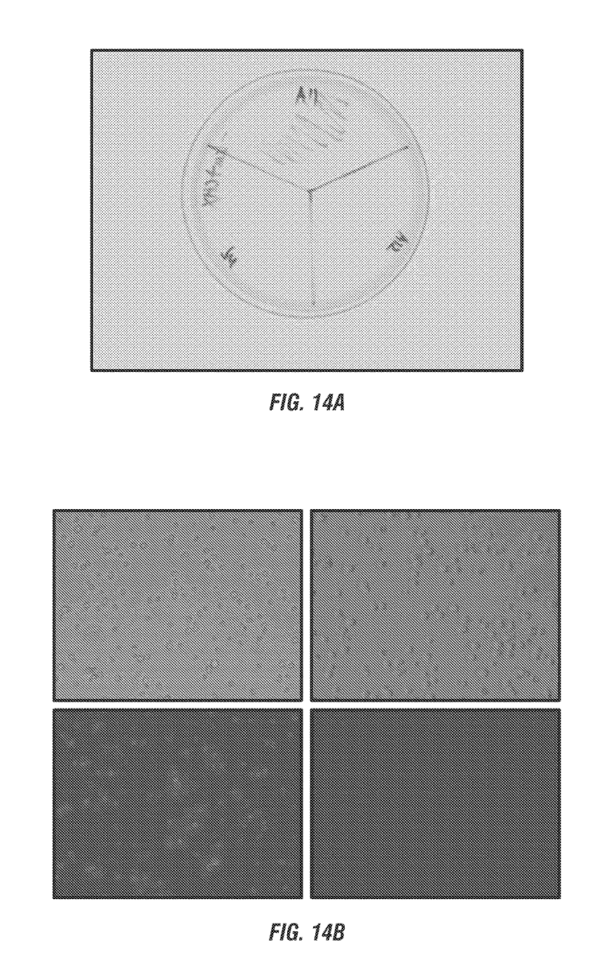

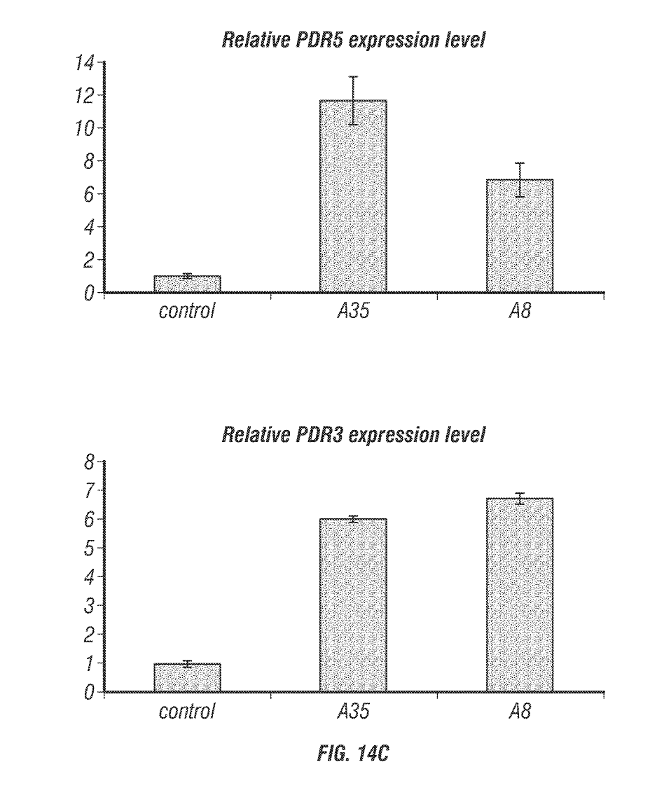

The inventors again tested the effectiveness of the TALE-based one-hybrid screening approach for microRNA gene targets. Specifically, the inventors used part of the promoter sequence of human miR-34b/c gene as the bait sequence and isolated 4 positive clones, which were confirmed by re-streaking on Aureobasidin A-containing plates (FIG. 14a). The RVD sequences of these 4 clones were extracted and two of them (M1, M17) are predicted to target the same target sequence within the miR-34b/c promoter (FIG. 14b). To confirm this TALE-DNA target binding, the inventors similarly generated baits which exclude those predicted DNA target sites. While the isolated TALE-VP16 fusions could induce expression of Aureobasidin A resistance gene when the bait sequence was intact (FIG. 14c, left), it failed to do so when the DNA target site was removed (FIG. 14c, right). The TALE-VP16 M1 clone was then cloned into a mammalian expression vector and after being transiently transfected into HEK293 or HeLa cells, the expression levels of miR-34b were measured by quantitative RT-PCR. As illustrated in FIG. 14c, TALE-VP16 M1 fusion successfully induced the expression levels of miR-34b in both cell lines. These results demonstrated that the inventors' TALE-VP16 library can be used to isolate TALEs with highest binding affinities to any DNA target sequences.

### Example 5

### TALE-VP16 Library for Positive Genetic Screening in Yeast Cells

The inventors applied the TALE-VP16 library to screen for TALE-VP 16 fusion proteins which confer resistance to cycloheximide in yeast. The inventors choose this specific phenotypic screening for two reasons. First, multidrug resistance has increasingly become a serious condition during the treatment of many infectious diseases. For example, yeast such as Candida species could become resistant under long term treatment with azole preparations. Secondly, relatively abundant knowledge has been available to part of the underlying mechanisms for multidrug resistance. For example, in the yeast S. cerevisiae, overexpression of ATP-binding cassette (ABC) transporters such as Pdr5p has been shown to contribute to cycloheximide resistance. In addition, the expression of PDR5 gene was known to be positively regulated by two homologous zinc finger-containing transcription regulators, Pdr1p and Pdr3p. The inventors expect that the screening could shed light on novel proteins/pathways which may be involved in multidrug resistance, as well as corroborate current known gene targets, such as PDR3 or PDR5.

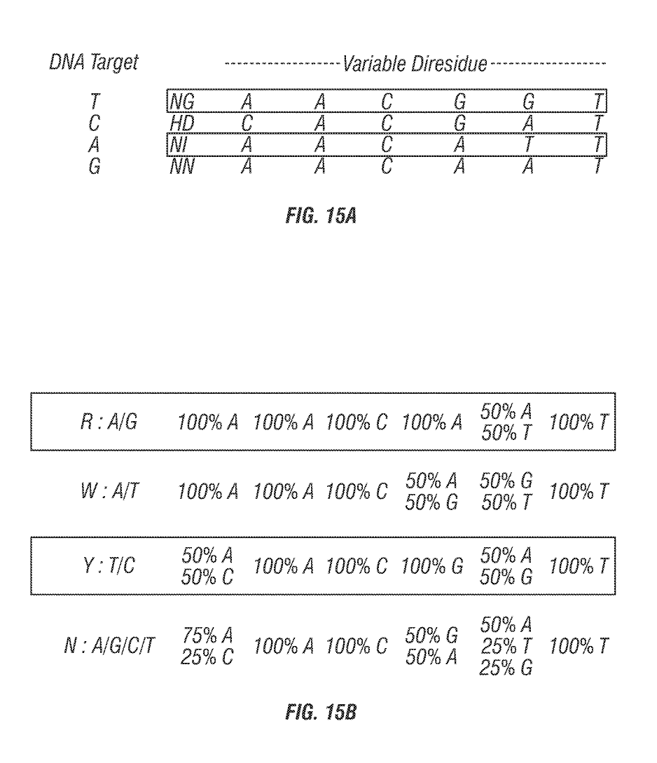

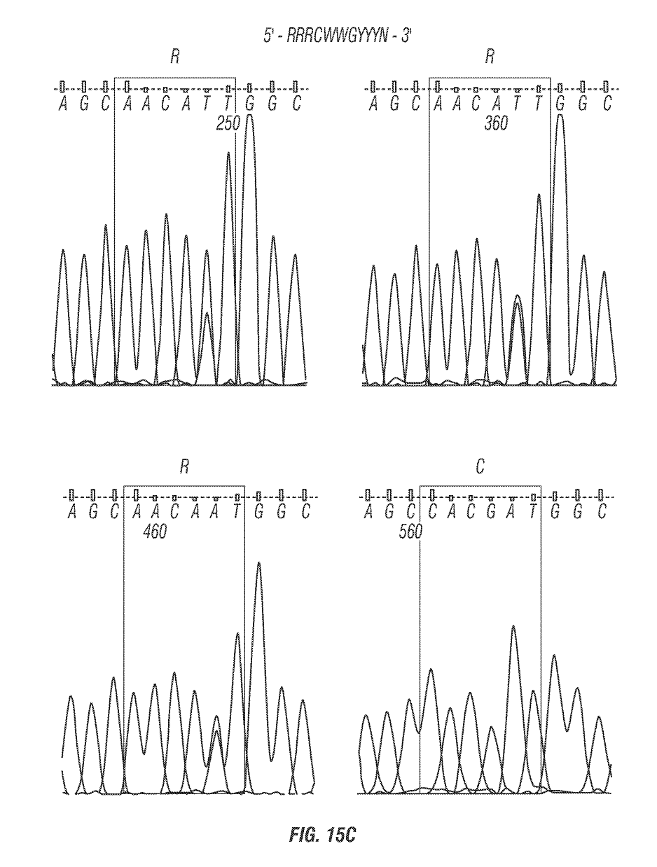

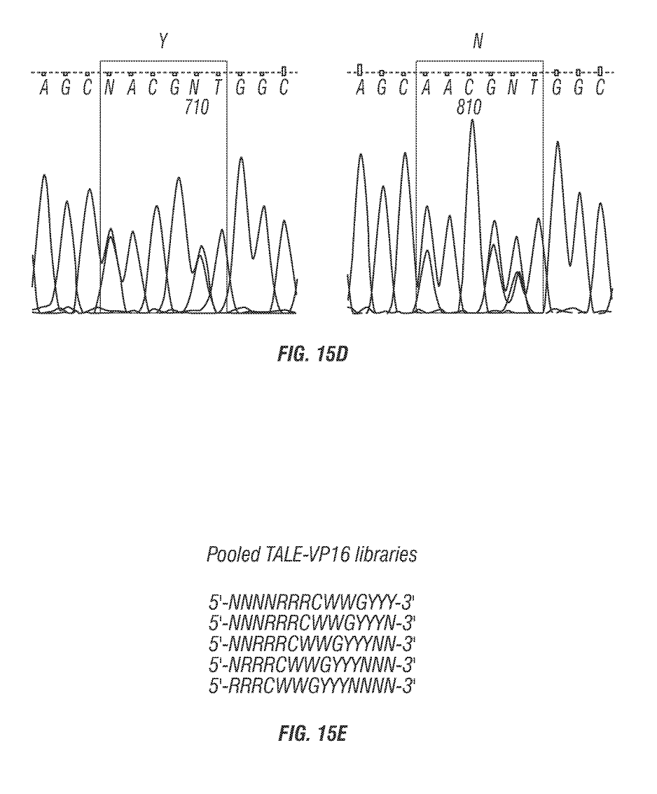

The inventors first determined the cycloheximide working concentration (0.4 ug/ml) for the screening assay, which was the lowest concentration at which the wild-type yeast cells (S. cerevisiae, strain name: Y1HGold, Clontech) fail to grow during the experimental period (96 hours). The inventors then applied the TALE-VP16 library and isolated 18 positive clones which can tolerate the presence of cycloheximide. Subsequently, the inventors isolated these TALE-VP16 fusion plasmids and re-transformed them back to the wild-type cells for confirmation, as in the original screening step, the natural mutations (both gain-of-function and loss-of-function) of yeast genome could artificially increase the cells' resistance to cycloheximide. Five (5) genuine positive clones were confirmed (FIG. 15a), isolated and sequenced to extract the TALE RVD sequences (FIG. 15b). Interestingly, two clones (A8, A35) are predicted to target the promoter of PDR3 gene and in addition, A35 is also predicted to bind to the promoter of PDR5 gene. Two methods were used to confirm these observations. First, the inventors prepared yeast cells transformed with TALE-VP16 fusions A8, A35 or pGADT7 empty vector and measured the expression levels of PDR3/PDR5 by quantitative RT-PCR (FIG. 15c). Indeed, both clones are able to effectively induce the overexpression of both PDR3 and PDR5. It is interesting to note that clone A35 showed a higher induction rate of expression of PDR5, possible due to the fact that compared to clone A8, it may also directly bind to the promoter of PDR5 gene. Secondly, the inventors cloned four copies of the predicted PDR3/PDR5 promoter target sequences in front of a fluorescence reporter gene (mKATE2) in yeast (bait). The inventors then transformed these yeast stable cells with either according A8/A35 TALE-VP16 fusions or pGADT7 (control). As illustrated in FIG. 15d, in contrast to the control, TALE-VP16 fusion clones A8 or A35 can potently induced the expression of mKATE2, further proving that these two fusions are able to efficiently target the promoters of PDR3 and PDR5 genes.

### Example 6

### Construction of a 11-Mer TALE Library for Negative Screening in Yeast Cells

To construct a TALE suppressor library, the inventors fuse the TALE DNA binding domain with two yeast suppressor domains, Tup1 or the C-terminal domain of Stc1. First, the general transcriptional repressor Tup1 forms a transcriptional co-repressor complex with Ssn6p. And its suppression mechanisms include the interaction with RNA polymerase II holozenzyme components and the alteration of chromatin structure through interaction with histones H3 and H4 and histone deacetylases. In the inventors' design, the inventors fuse with either the N terminus 1-201aa, which has been successfully used in a library screening, or the full-length protein. Secondly, it is recently reported that the the C-terminal region of Stc1 mediates association with Clr4 complex (CLRC), which subsequently regulates methylation of histone H3 on lysine 9 (H3K9me) in cognate chromatin and induces gene silencing. Two methods can be used to construct these TALE fusion libraries. First, while assembling pFUS_A and pFUS_B into the final products, the inventors will use a pTAL backbone plasmid which harbors a Tup1 or Stc1 suppresor domain. An alternative and possibly more efficient way is to take advantage of the homologous recombination reactions (SMART technology, Clontech) during yeast transformation. In this case, the inventors will remove the pre-existing Gal4 activation domain and introduce the Tup1 or Stc1 repressor domain downstream of CDS III homologous sequence on the prey plasmid (pGADT7-Rec, Clontech). In addition, the inventors will design PCR primers which will only amplify the DNA binding domain of the existing 11-mer TALE-VP16 library. The design will also ensure the downstream suppressor domains are in frame with the DNA binding domain so the fusions could be properly translated. The major advantage for this approach lies in that it utilizes the already-made TALE-VP16 library and circumvents the task of preparation of new plasmid libraries, which are time-consuming and expensive. This TALE-based suppression library can then be used for negative genetic screening, as detailed previously.

### Example 7

### Construction of a Virus-Based 11-Mer TALE-BP16 Viral Library for Positive Screening in Human Cells

The inventors envisioned using these libraries for genome-wide phenotype screens in human cells. Accordingly, they have completed the initial steps along this direction using an adeno-associated viral system (Agilent Technologies). First, the ORFs of a complete 11-mer TALE-VP16 library were amplified from the original vectors and subsequently cloned into the AAV-MCS vector. As the ORFs differ minimally (i.e., RVD sites) the library fidelity was preserved during this step. The inventors confirmed the results using standard Sanger sequencing with primers flanking the TALE DNA binding domain. We then proceeded with the preparation of the 11-mer TALE-VP16 AAV viral stocks using the AAV helper-free system.

Two methods were used to confirm the functional integrity of these TALE-based AAV viral libraries. First, the inventors probed the efficiency of TALE delivery using AAVs. HEK293 cells were infected with the viral library at a fixed MOI of 400 and in parallel cells were transiently transfected with variable amount of the corresponding AAV-TALE-VP16 plasmid library. The relative expression of TALE-VP16 mRNAs was measured by quantitative RT-PCR using primers for the VP16 domain. The results show that the infection of TALE-based AAV viral stock at MOI 400 was equivalent to transient transfection of approximately 16.25 ng of plasmid.

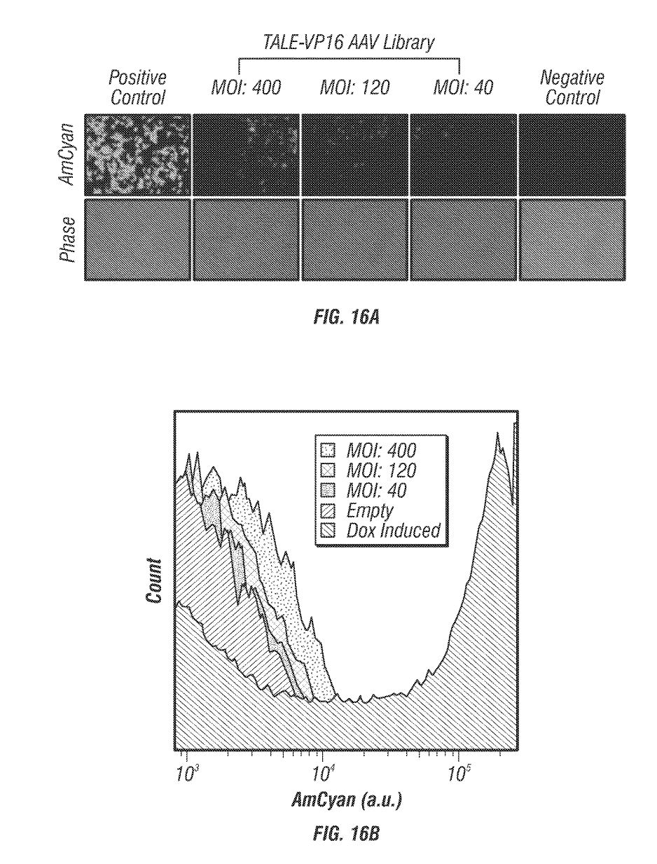

Second, the inventors infected with the AAV viral library, at a range of MOIs (400, 120, 40 and 0), an established HEK293 stable cell line which harbors an amCyan fluorescent reporter gene under the control of a tetracycline responsive element (TRE) and minimum CMV promoter. The particular cell line also contains the reverse tetracycline-controlled transactivator (rtTA) protein transcript under the control of a CMV promoter. In the presence of doxycycline, rtTA binds the TRE element and drives the expression of amCyan. 48 hours post-infection, both fluorescence microscopy images and flow cytometry data demonstrate the activation of amCyan in a subpopulation of the HEK293 cells in a MOI-dependent manner, indicating that cells received TALE-VP16 fusions which bind to the TRE site or the ORF of amCyan (FIG. 16).

### Example 8

### Construction of a 11-Mer TALE-VP16 Library for Methylated DNA Target Sequences in Human Cells

Hypermethylation of the promoter region, which often results in the silencing of its downstream gene, is a common feature in mammalian cells, and plays critical roles in various functions such as development, differentiation, and tumorigenesis. This phenomenon presents a challenge for the screening methods as the TALE RVD HD does not bind to methylated cytosine. Recently, it is reported that RVD H* (the asterisk indicates that amino acid 13 is missing) is able to effectively target methylated cytosine (5mC), in addition to thymidine (T) but not unmethylated cytosine. Based on this important finding, the inventors will also construct a TALE-VP16 library which also includes the basic RVD building element H*. In essence, as illustrated in FIG. 11c, the inventors will use equal amount of NN, NI, NG, HD and H* in the golden gate assembling reactions. The resulting TALE-VP16 cDNAs will then be subsequently cloned into a AAV delivery system as describe in above. The inventors expect this library to be able to effectively bind to methylated DNA sequences. More importantly, since H* displays differential binding affinities between methylated and unmethylated cytosine, this library could also be used to specifically target hypermethylated promoter sequences, which are frequently observed in cancer cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bartel, MicroRNAs: target recognition and regulatory functions. Cell, 136:215-233, 2009.
Benenson, Biomolecular computing systems: principles, progress and potential. Nat Rev Genet, 13:455-468, 2012.
Bleris et al., Synthetic incoherent feedforward circuits show adaptation to the amount of their genetic template. Mol Syst Biol, 7:519, 2011.
Boch et al., Breaking the code of DNA binding specificity of TAL-type III effectors. Science, 326:1509, 2009.
Boch and Bonas, Xanthomonas AvrBs3 family-type III effectors: Discovery and function. Annu Rev Phytopatholi, 48:419-436, 2010.
Bochtler, Structural basis of the TAL effector-DNA interaction, Bio Chem, 393:1055-66, 2012.
Bogdanove and Voytas, TAL Effectors: Customizable Proteins for DNA Targeting, Science, 333:1843-1846, 2011.
Boyle and Silver, Parts plus pipes: Synthetic biology approaches to metabolic engineering. Metab Eng, 14:223-32, 2011.
Bradley, Structural modeling of TAL effector-DNA interactions. Protein Science, 21:471-4, 2012.
Briggs et al., Iterative capped assembly: rapid and scalable synthesis of repeat-module DNA such as TAL effectors from individual monomers. Nucleic Acids Res, 40:e117, 2012.
Carlson et al., Targeting DNA With Fingers and TALENs, Mol Ther Nucleic Acids, 1:e3, 2012.
Cermak et al., Efficient design and assembly of custom TALEN and other TAL effector-based constructs for DNA targeting. Nucleic Acids Res, 39:E82, 2011.
Certo et al., Tracking genome engineering outcome at individual DNA breakpoints, Nature methods, 8:671-676, 2011.
Chang et al., Dissection of the LXXLL nuclear receptor-coactivator interaction motif using combinatorial peptide libraries: discovery of peptide antagonists of estrogen receptors α and β. Mol Cell Biol, 19:8226-8239, 1999.
Christi, Biodiesel from Microalgae, Biotechnol Adv, 25:294-306, 2007.
Christian et al., Targeting DNA double-strand breaks with TAL effector nucleases. Genetics, 186:757-761, 2010.
Clomburg and Gonzalez, Biofuel production in Escherichia coli: the role of metabolic engineering and synthetic biology, Appl Microbiol Biotechnol, 86:419-434, 2010.
Cong et al., Comprehensive interrogation of natural TALE DNA-binding modules and transcriptional repressor domains. Nat Commun, 3:968, 2012.
Deng et al., Structural basis for sequence-specific recognition of DNA by TAL effectors. Science, 335:720-723, 2012.
Deng et al., Recognition of methylated DNA by TAL effectors, Cell Res, 22:1502-4, 2012.
Ding et al., A TALEN Genome-Editing System for Generating Human Stem Cell-Based Disease Models, Cell Stem Cell, 12:238-51, 2012.
Doyle et al., TAL Effector-Nucleotide Targeter (TALE-NT) 2.0: tools for TAL effector design and target prediction. Nucleic Acids Res, 40:W117-W122, 2012.
Gabsalilow et al., Site- and strand-specific nicking of DNA by fusion proteins derived from MutH and I-SceI or TALE repeats, Nucleic Acids Res, 41:e83, 2013.
Gao et al., Crystal structure of a TALE protein reveals an extended N-terminal DNA binding region, Cell Res, 22:1716-20, 2012.
Garg et al., Engineering synthetic TAL effectors with orthogonal target sites. Nucleic Acids Res, 40:7584-7595, 2012.
Geiβler et al., Transcriptional activators of human genes with programmable DNA-specificity, PLoS One, 6:e19509, 2011.
Grindley et al., Mechanisms of Site-Specific Recombination, Annu Rev Biochem, 75:567-605, 2006.
Gürlebeck et al., Dimerization of the bacterial effector protein AvrBs3 in the plant cell cytoplasm prior to nuclear import The Plant Journal, 42:175-187, 2005.
Hartlerode and Scully, Mechanisms of double-strand break repair in somatic mammalian cells. Biochem J, 423:157, 2009.
Hockemeyer et al., Genetic engineering of human pluripotent cells using TALE nucleases, Nat Biotechnol, 29:731-734, 2011.
Holtz and Keasling, Engineering static and dynamic control of synthetic pathways. Cell, 140:19-23, 2010.
Kay et al., Characterization of AvrBs3-like effectors from a Brassicaceae pathogen reveals virulence and avirulence activities and a protein with a novel repeat architecture, Mol Plant-Microbe Interact, 18:838-848, 2005.
Kay et al., A bacterial effector acts as a plant transcription factor and induces a cell size regulator, Science, 318:648, 2007.
Keasling, Synthetic biology for synthetic chemistry, ACS Chemical Biology, 3:64-76, 2008.
Keasling, Synthetic biology and the development of tools for metabolic engineering, Metab Eng, 14:189-95, 2012.
Khanna and Jackson, DNA double-strand breaks: signaling, repair and the cancer connection, Nat Genet, 27:247-254, 2001.
Kim et al., Surrogate reporters for enrichment of cells with nuclease-induced mutations. Nat Methods, 8:941-943, 2011.
Kim et al., A library of TAL effector nucleases spanning the human genome, Nat Biotechnol, 31:251-8, 2013.
Kleinstiver et al., Monomeric site-specific nucleases for genome editing. Proc Natl Acad Sci USA, 109:8061, 2012.
Lee et al., Metabolic engineering of microorganisms for biofuels production: from bugs to synthetic biology to fuels, Curr Opin Biotechnol, 19:556-563, 2008.
Lee and Lee, Mammalian two-hybrid assay for detecting protein-protein interactions in vivo. Methods Mol Biol, 439:327, 2008.
Li et al., TAL nucleases (TALNs): hybrid proteins composed of TAL effectors and Fold DNA-cleavage domain. Nucleic Acids Res, 39:359-372, 2011.
Li et al., Modularly assembled designer TAL effector nucleases for targeted gene knockout and gene replacement in eukaryotes, Nucleic Acids Res, 39:6315-6325, 2011b.
Li et al., Transcription activator-like effector hybrids for conditional control and rewiring of chromosomal transgene expression, Scientific Reports, 2, 2012.
Li et al., Rapid and highly efficient construction of TALE-based transcriptional regulators and nucleases for genome modification, Plant Mol Biol, 78:407-16, 2012.
Lievens et al., Mammalian two-hybrids come of age. Trends Biochem Sci 34:579-588, 2009.
Long et al., Protein kinase C modulates aryl hydrocarbon receptor nuclear translocator protein-mediated transactivation potential in a dimer context. J Biol Chem, 274:12391, 1999.
Maeder et al., Robust, synergistic regulation of human gene expression using TALE activators, Nature methods, 10:243-245, 2013.
Mahfouz et al., De novo-engineered transcription activator-like effector (TALE) hybrid nuclease with novel DNA binding specificity creates double-strand breaks, Proceedings of the National Academy of Sciences, 108:2623, 2011.
Mahfouz and Li, TALE nucleases and next generation GM crops, GM Crops, 2, 2011b. Mak et al., The crystal structure of TAL effector PthXo1 bound to its DNA target. Science, 335:716-719, 2012.
Mak et al., The Crystal Structure of TAL Effector PthXo1 Bound to Its DNA Target, Science, 335:716-9, 2012.
Maresca et al., Obligate Ligation-Gated Recombination (ObLiGaRe): Custom designed nucleases mediated targeted integration through non-homologous end joining, Genome Res, 2012. Marx, Genome-editing tools storm ahead, Nature Methods, 9:1055-1059, 2012.
Matsuda and Cepko, Electroporation and RNA interference in the rodent retina in vivo and in vitro. Proc Natl Acad Sci USA, 101:16, 2004.
Mercer et al., Chimeric TALE recombinases with programmable DNA sequence specificity, Nucleic Acids Res, 2012.
Miller et al., A TALE nuclease architecture for efficient genome editing, Nat Biotechnol, 29:143-148, 2010.
Morbitzer et al., Regulation of selected genome loci using de novo-engineered transcription activator-like effector (TALE)-type transcription factors, Proc Natl Acad Sci USA, 107:21617, 2010.
Moscou and Bogdanove, A simple cipher governs DNA recognition by TAL effectors. Science, 326:1501-1501, 2009.
Muñoz Bodnar et al., Tell Me a Tale of TALEs, Mol Biotechnol, 53:228-35, 2012.
Peng et al., Biochemical analysis of the Kruppel-associated box (KRAB) transcriptional repression domain, J Biol Chem, 275:18000-18010, 2000.
Pennisi, The Tale of the TALEs, Science, 338:1408-1411, 2012.
Politz et al., Artificial repressors for controlling gene expression in bacteria, Chemical Communications, 49:4325-7, 2013.
Ramirez et al., Engineered zinc finger nickases induce homology-directed repair with reduced mutagenic effects, Nucleic Acids Res, 40:5560-5568, 2012.
Reyon et al., ZFNGenome: a comprehensive resource for locating zinc finger nuclease target sites in model organisms, BMC Genomics, 12:83, 2011.
Reyon et al., FLASH assembly of TALENs for high-throughput genome editing, Nat Biotechnol, 30:460-465, 2012.
Rinaudo et al., A universal RNAi-based logic evaluator that operates in mammalian cells. Nat Biotechnol, 25:795-801, 2007.
Ruder et al., Synthetic biology moving into the clinic. Science, 333:1248-1252, 2011.
Sanjana et al., A transcription activator-like effector toolbox for genome engineering, Nature protocols, 7:171-192, 2012.
Schmid-Burgk et al., A ligation-independent cloning technique for high-throughput assembly of transcription activator-like effector genes, Nat Biotechnol, 31:76-81, 2012.
Schornack et al., Characterization of AvrHah1, a novel AvrBs3-like effector from Xanthomonas gardneri with virulence and avirulence activity, New Phytol, 179:546-556, 2008.
Shiue and Prather, Synthetic biology devices as tools for metabolic engineering, Biochem Eng J, 2012.
Stegmeier et al., A lentiviral microRNA-based system for single-copy polymerase II-regulated RNA interference in mammalian cells. Proc Natl Acad Sci USA, 102:13212, 2005.
Streubel et al., TAL effector RVD specificities and efficiencies. Nat Biotechnol, 30:593-595, 2012.
Sugio et al., Two type III effector genes of Xanthomonas oryzae pv. oryzae control the induction of the host genes OsTFIIAγ1 and OsTFX1 during bacterial blight of rice. Proc Natl Acad Sci USA, 104:10720, 2007.
Sun et al., Optimized TAL effector nucleases (TALENs) for use in treatment of sickle cell disease. Mol. BioSyst., 8:1255-63, 2012.
Suzuki and Bird, DNA methylation landscapes: provocative insights from epigenomics, Nature Reviews Genetics, 9:465-476, 2008.
Tesson et al., Knockout rats generated by embryo microinjection of TALENs, Nat Biotechnol, 29:695-696, 2011.
Tong et al., Rapid and Cost-Effective Gene Targeting in Rat Embryonic Stem Cells by TALENs, Journal of Genetics and Genomics, 39:275-80, 2012.
Tran et al., Production of unique immunotoxin cancer therapeutics in algal chloroplasts, Proceedings of the National Academy of Sciences, 110:E15-E22, 2013.
Tremblay et al., TALE proteins induced the expression of the frataxin gene. Hum Gene Ther, 23:883-90, 2012.
Tyo and Alper, Stephanopoulos GN. Expanding the metabolic engineering toolbox: more options to engineer cells, Trends Biotechnol, 25:132-137, 2007.
Valton et al., Overcoming TALE DNA Binding Domain Sensitivity to Cytosine Methylation, J Biol Chem, 287:38427-32, 2012.
Wefers et al., Direct production of mouse disease models by embryo microinjection of TALENs and oligodeoxynucleotides. Proc Natl Acad Sci USA, 110:3782-7, 2013.
Wijnhoven et al., MicroRNAs and cancer. Br J Surg, 94:23-30, 2007.
Xie et al., Multi-input RNAi-based logic circuit for identification of specific cancer cells. Science, 333:1307, 2011.
Yonekura-Sakakibara et al., Transcriptome data modeling for targeted plant metabolic engineering, Curr Opin Biotechnol, 24:285-90, 2012.
Yuan et al., Cobalt inhibits the interaction between hypoxia-inducible factor-α and von Hippel-Lindau protein by direct binding to hypoxia-inducible factor-α. J Biol Chem, 278:15911, 2003.
Zhang et al., Efficient construction of sequence-specific TAL effectors for modulating mammalian transcription, Nat Biotechnol, 29:149-153, 2011.
Zhao et al., A coumermycin/novobiocin-regulated gene expression system. Hum Gene Ther, 14:1619-1629, 2003.

What is claimed is:

1. A method of preparing a random N-mer transcription activator-like effector (TALE) library, the method comprising:
(a) generating N populations of DNA binding repeats, each comprising repeat variable diresidues (RVDs) flanked by an upstream and a downstream sequence for BsaI-based digestion, wherein the upstream and the downstream flanking sequences for Bsa1-based digestion are unique for each population;
(b) digesting the N populations of DNA binding repeats with Bsa1, wherein the resulting 3′ overhang of a first population of DNA binding repeats is complementary to the resulting 5′ overhang of a second population of DNA binding repeats;
(c) digesting a plasmid with Bsa1, wherein the resulting 3′ overhang is complementary to the 5′ overhang of the first population of DNA binding repeats and the 5′ overhang is complementary to the 3′ overhang of the N^{th} population of DNA binding repeats; and
(d) ligating the digested N populations of DNA binding repeats into the digested plasmid, thereby preparing a random N-mer TALE library.

2. The method of claim 1, further comprising:
(e) replicating the plasmids within a population of host cells;
(f) isolating plasmid DNA from the population of host cells; and
(g) pooling the isolated plasmid DNA.

3. The method of claim 1, wherein the RVDs in each population of DNA binding repeats are present in an equal ratio and wherein each module has an equal chance of incorporation.

4. The method of claim 3, wherein the random N-mer TALE library is further defined as a balanced library targeting all possible combinations with equal probability.

5. The method of claim 1, wherein the RVDs in each population of DNA binding repeats are present in an unequal ratio.

6. The method of claim 5, wherein the random N-mer TALE library is further defined as a nucleotide-biased library.

7. The method of claim 6, wherein the nucleotide-biased library is a GC-biased library.

8. The method of claim 6, wherein the nucleotide-biased library is a AT-biased library.

9. The method of claim 1, wherein select populations of DNA binding repeats comprise a single RVD.

10. The method of claim 5 or 9, wherein the random N-mer TALE library is further defined as a sequence-biased library.

11. The method of claim 1, wherein the RVDs determine the recognition of a base in the target DNA sequence, wherein each DNA binding repeat is responsible for recognizing one base in the target DNA sequence, and wherein each RVD comprises a member selected from the group consisting of:
NG for recognizing T;
HD for recognizing C;
NI for recognizing A;
NN for recognizing G; and
H* for recognizing methylated cytosine (5mC), wherein the * indicates that the second amino acid in the RVD is deleted.

12. The method of claim 1, wherein N is at least 10.

13. The method of any one of claim 1, 6, or 10, wherein the random N-mer TALE library is fused to a nucleotide sequence coding for a functional domain.

14. The method of claim 1, wherein the functional domain is a transcription regulatory domain, nuclease, integrase, or nickase.

15. The method of claim 14, wherein the transcription regulatory domain is a transcription activator.

16. The method of claim 14, wherein the transcription regulatory domain is a transcription repressor.

17. The method of claim 1, wherein the plasmids are viral vectors and the library is a viral library.

18. A method of determining a TALE that binds to a given nucleotide sequence comprising:
(a) obtaining a random N-mer TALE library of claim 15;
(b) expressing the library in a population of cells that comprise a reporter gene operably linked to a promoter comprising the given nucleotide sequence, wherein expression of the reporter gene is dependent on the presence of a TALE-transcription activator fusion that can bind to the given nucleotide sequence;
(c) selecting for cells that express the reporter gene;
(d) isolating plasmid DNA from the selected cells; and
(e) sequencing the plasmid DNA to determine the sequence of the TALE that bound the given nucleotide sequence.

19. The method of claim 18, wherein the given nucleotide sequence is a promoter.

20. The method of claim 19, wherein the promoter is an endogenous human promoter.

21. A method of performing a genetic screen comprising:
(a) obtaining a random N-mer TALE library of claim 13;
(b) expressing the library if step (b) in a population of cells;
(c) selecting for cells with a desired phenotype;
(d) isolating plasmid DNA from the selected cells; and
(e) sequencing the plasmid DNA to determine the sequence of the TALE-fusion that imparted the desired phenotype.

22. The method of claim 21, wherein the genetic screen is performed in yeast.

23. The method of claim 22, wherein the genetic screen is a positive genetic screen.

24. The method of claim 22, wherein the genetic screen is a negative genetic screen.

25. The method of claim 21, wherein the screen is performed in human cells.

26. The method of claim 25, wherein the screen is a methylation-based genetic screen.

27. The method of claim 21, wherein the screen is performed for production of induced pluripotent stem cells.

28. A random N-mer TALE library produced according to claim 1.

29. A population of host cells comprising a random N-mer TALE library of claim 1.
